(19) United States
(12) Patent Application Publication
(10) Pub. No.: US 2022/0323412 A1
(43) Pub. Date: Oct. 13, 2022

(54) EXTRACELLULAR VESICLE-FENRETINIDE COMPOSITIONS, EXTRACELLULAR VESICLE-C-KIT INHIBITOR COMPOSITIONS, METHODS OF MAKING AND USES THEREOF

(71) Applicants: Chi-Chih Kang, San Francisco, CA (US); Ishai Padawer, San Francisco, CA (US); Pranali Prabhakar Deshpande, San Francisco, CA (US); Catherine Rosemary Planey, San Francisco, CA (US); Alexander Fen Mok, San Francisco, CA (US); Zachary Swinney, San Francisco, CA (US)

(72) Inventors: Chi-Chih Kang, San Francisco, CA (US); Ishai Padawer, San Francisco, CA (US); Pranali Prabhakar Deshpande, San Francisco, CA (US); Catherine Rosemary Planey, San Francisco, CA (US); Alexander Fen Mok, San Francisco, CA (US); Zachary Swinney, San Francisco, CA (US)

(21) Appl. No.: 17/640,749

(22) PCT Filed: Sep. 8, 2020
(86) PCT No.: PCT/US2020/049774

§ 371 Date: Mar. 4, 2022

(51) Int. Cl. A61K 31/404; A61K 9/127

(52) U.S. Cl. CPC A61K 31/404; A61K 9/127

(57) ABSTRACT

Provided herein is a method of preparation of a therapeutic formulation comprising extracellular vesicles packaged with fenretinide.

### CROSS REFERENCE TO RELATED APPLICATIONS

This application claims benefit to U.S. Provisional Application No. 62/897,211, filed on Sep. 6, 2019, and U.S. Provisional Application No. 62/935,530, filed on Nov. 14, 2019, which are incorporated herein by reference in their entireties for all purposes. All publications, patents, and patent applications mentioned in this specification are herein incorporated by reference to the same extent as if each individual publication, patent, or patent application was specifically and individually indicated to be incorporated by reference.

### BACKGROUND OF THE INVENTION

4-HPR (Fenretinide; 15-[(4-hydroxyphenyl)amino]retinal; N[4-hydroxyphenyl]retinamide; CAS No.: 65646-68-6) a synthetic analog of Retinoic Acid (RA) that mediates the functions of vitamin A for growth and development. RA can bind to the Retinoic Acid Receptor (RAR), which can alter the conformation of RAR and can affect the binding of other proteins to RAR that induce or repress transcription of nearby genes, such as the Hox genes (Szatmari, I et al. Stem Cells. 2010 September; 28(9): 1518-1529. doi: 10.1002/stem.484). In addition to RAR-dependent regulatory mechanism, 4-HPR and its metabolites are known to have RAR-independent effects (Sheikh, M et al. (1995) Carcinogenesis 16:2477-2486; Fanjul, A et al. (1996) J Biol Chem 271:22441-22446; Kazmi, Set al. (1996) Cancer Res 56:1056-1062; Clifford, J L et al. (1999) Cancer Res 59:14-18; Sabichi, A et al. (2003) Clin Cancer Res 9:4606-4613; Moise, A R et al. (2007) Biochem 46:4449-4458; Bouriez, D (2018) Int J Mol Sci 19:3388; Mcilroy G D et al. (2016) Biochem Pharm 100:86-97).

4-HPR (Fenretinide) can be highly insoluble in water with a solubility limit of about 1.71 μg/mL (Orienti et al. (2019) Cell Death Disease 10:529) and a logP of greater than about 6 (Wishart, D S et al. Nucleic Acids Res (2018) 46:D1074-D1082; see DrugBank Accession Number DB05076).

Fenretinide is usually suspended in corn oil and polysorbate 80 for clinical formulation (Cooper, J P et al. Exp Biol Med (Maywood). 2017 June; 242(11): 1178-1184. Published online 2017 Apr. 21. doi: 10.1177/1535370217706952). There is currently no indication in the United States where fenretinide is a first-line treatment. This may be due to several factors, including low bioavailability and/or poor patient compliance in taking the required number of capsules (Cooper, J Pet al. Exp Biol Med (Maywood). 2017 Jun; 242(11): 11781184. Published online 2017 Apr. 21. doi: 10.1177/1535370217706952) Clinical trials aimed at evaluating the activity of fenretinide in cancer patients yielded frustrating results as therapeutic plasma levels could not be attained due to the poor aqueous solubility and consequent low bioavailability of the drug.

c-KIT inhibitors are known and used to treat certain cancers. However, it is observed that over time, patients undergo c-KIT inhibitor resistance. Delivering c-KIT inhibitors with EVs, which are taken up by cells, could help boost the therapeutic effects of c-KIT inhibitors in these scenarios.

There is a need for improved fenretinide compositions and uses thereof to, e.g., increase systemic Fenretinide exposure with an aim to improve clinical outcomes. In addition, a need exists for improved composition for c-kit inhibitors so as to increase intracellular delivery of c-kit inhibitors in, e.g., cancer patients experiencing c-kit inhibitor resistance, and target intracellularly localized mutant c-kit receptors.

### SUMMARY OF THE INVENTION

Provided herein is a method of treating macular degeneration comprising administering to a subject in need thereof a therapeutic formulation comprising an extracellular vesicle comprising fenretinide. Provided herein is a method of treating cancer. In one embodiment, the method comprises administering to a subject in need thereof a therapeutic formulation comprising an extracellular vesicle comprising fenretinide. In another embodiment, the method comprises administering to a subject in need thereof a therapeutic formulation comprising an extracellular vesicle comprising a c-KIT inhibitor.

Also, the invention provides methods of preparing and purifying an extracellular vesicle(s) comprising fenretinide or, alternatively, a c-KIT inhibitor. In an embodiment of the invention, the method comprises the step of incubating a suspension of extracellular vesicles (EV) with a solution comprising fenretinide or a c-KIT inhibitor in order to prepare an EV-fenretinide formulation comprising extracellular vesicles comprising fenretinide or a c-KIT inhibitor formulation comprising extracellular vesicles comprising a c-KIT inhibitor, respectively. The method further comprises the step of purifying the extracellular vesicles comprising fenretinide or a c-KIT inhibitor from fenretinide or a c-KIT inhibitor, respectively, which not packaged in an EV. Additionally provided herein are methods for purifying the extracellular vesicles comprising fenretinide or a c-KIT inhibitor away from fenretinide or a c-KIT inhibitor, respectively, which is not packaged in an EV(s).

Further provided herein are compositions comprising extracellular vesicles including fenretinide. Additionally, the invention provides compositions comprising extracellular vesicles having c-KIT inhibitors. The compositions maybe in various formulations such as for oral administration, intravenous administration, or intravitreal administration.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows absorption spectra for fenretinide-EV, EV (alone) and fenretinide (alone) samples obtained after subjecting to the same clean-up protocol to eliminate soluble fenretinide free of EV and fenretinide precipitates due to poor solubility of fenretinide in an aqueous environment.

FIGS. 2A and 2B show the linear trends of 375 nm/260 nm absorption ratios of purified fenretinide-EV obtained after incubating a fixed amount of EVs with varying fenretinide concentration in ddH₂O or 20 mM Tris (about pH 6.5).

FIGS. 3A and 3B show viability of MCF breast cancer cells as a function of fenretinide concentration and EV particle, respectively, following 48-hr treatment with fenretinide-EV (in phosphate buffer saline, PBS), fenretinide alone (solubilized in 100% DMSO), EV alone (in PBS) and vehicle (DMSO) count using CellTiter-Glo™ Luminescent Cell Viability Assay.

FIGS. 4A and 4B show the Fenretinide concentration over time and in tissue, respectively, as quantified by LC-MS from blood and tissue collections of CD-1® IGS mice (Charles River) injected with EV-Fenretinide and Fenretinide alone formulation (Fenretinide). Both Fenretinide-EV and Fenretinide were animal weight normalized at a dosing level of 0.2288 mg/kg (i.e., a dose of 5.72 μg fenretinide to an about 25 g CD-1® IGS mouse).

FIG. 5 shows the toxicology (blood count) data of CD-i® IGS mice 16-hr after intravenous injection of Fenretinide alone or Fenretinide-EV. Both Fenretinide-EV and Fenretinide were animal weight normalized at a dosing level of 0.2288 mg/kg (i.e., a dose of about 5.72 μg fenretinide to a/25 g CD-1® IGS mouse). As reference, blood count of untreated CD-1® IGS mice obtained from Charles River is provided [https://www.criver.com/sites/default/files/resources/CD-1IGSMouseModelInformationSheet.pdf].

FIG. 6 shows the stability of the fenretinide-EV formulation after freezing and storage at −80° C. for 1, 7 or 14 days. The error bar in the inlet figure is SEM. n=6 independent Fenretinide-EV samples.

FIGS. 7A and 7B show transmission electron microscopy (TEM) images of uranyl acetate-stained samples comprising EV-only (“cup-shape” structure) and EV-Fenretinide (“walnut-like” structure), respectively. Scale bar=200 nm.

FIG. 8 shows the size exclusion chromatography profile of Fenretinide packaged EVs detected by an HPLC method developed to detect both Fenretinide and Cholesterol, the latter as a proxy for EVs.

FIGS. 9A and 9B show viability of a human retinal pigment epithelium cell line, ARPE-19 (ATCC® CRL-2302™), after treatment with fenretinide (resuspended in 100% DMSO) or fenretinide-EV (in PBS) at low (0.94 μM) or high (15 μM) active pharmaceutical ingredient (API) concentration, respectively. The error bar in the figure is SEM. n=3 independent wells.

FIG. 10 shows average percent retention of fenretinide in eyes 24-hrs after intravitreal injection of 5 uL 90.9 uM fenretinide alone or 5 uL of 94.8 uM fenretinide-EV formulation to each eye of a cohort of 130-150 g brown Norway rats (6 rats for each formulation). The error bar in the figure is SEM. n=6 rats.

FIG. 11 shows differences in eye biodistribution of fenretinide formulated as free fenretinide (excipient: 5% Ethanol, 12.5% Solutol-HS 15, 12.5% PEG 300, and 70% PBS) or fenretinide-EV (in PBS) 24-hrs after administration via intravitreal injection to each eye of brown Norway rat (6 rats/treatment condition). The error bar in the figure is SEM. n=6 rats.

FIG. 12 shows statistical analysis of differences between Fenretinide and Single Injection Fenretinide EV based on the average of the 2 eye values from each animal. Specifically, the figure shows the statistical analysis of differences in eye biodistribution of fenretinide formulated as free fenretinide (excipient: 5% Ethanol, 12.5% Solutol-HS 15, 12.5% PEG 300, and 70% PBS) or fenretinide-EV (in PBS) 24-hrs after administration via intravitreal injection to each eye of brown Norway rat (6 rats/treatment condition).

FIGS. 13A and 13B show reduced lesion size (neovascularization) and integrated density (vascular leakage) in a laser induced choroidal neovascularization (CNV) model of wet age-related macular degeneration after treatment with a single intravitreal injection fenretinide-EV formulation at a dose of about 5 uL, 94.8 uM Fenretinide-EV per eye. The lesion size and integrated density were quantified by fluorescein angiography (FA) on Day 22 after laser induced lesion in the Bruch's membrane. The error bar in the figure is SEM. n=28 lesions for vehicle control (PBS) and n=30 lesions for Fenretinide-EV.

### DETAILED DESCRIPTION OF THE INVENTION

Fenretinide can be a highly hydrophobic drug that can be difficult to deliver in vivo to animals and humans. In one aspect, provided herein is fenretinide formulated using Extracellular Vesicles (EVs) as a drug delivery vehicle. EVs can be incubated with fenretinide using e.g., PBS, followed by removal of fenretinide drug aggregates and clean up of free-floating fenretinide to obtain a highly pure, stable EV-fenretinide formulation. Free fenretinide can aggregate due to its poor water solubility, and thus specific protocol parameters have been optimized in order to avoid the fenretinide, e.g., all of the fenretinide, from aggregating and crashing out of solution before it can package with the EVs.

Delivering fenretinide using EVs can solve the clinical problems of low bioavailability and inability of enough fenretinide to reach its intracellular targets. When incubated under specific conditions, using specific buffers, merely by way of example, proteins on the surface of EVs can interact in a distinct manner to stably bind to fenretinide, and package fenretinide on the surface, partially or fully inside the lipid bilayer, or inside the EVs. This can allow a large amount of fenretinide to be delivered on each individual EV. EVs can be naturally taken up by cells and thus introduce fenretinide intracellularly, increasing interaction with the target (e.g., cells in the eye or cancer cells). This increase in available fenretinide at the desired areas can result in more durable treatment responses, more patients cured of a disease, and also reduce off-target side effects, such as toxicity, to improve patient quality of life.

The invention provides compositions having an isolated or enriched set of extracellular vesicles that contain fenretinide or a c-KIT inhibitor and a pharmaceutically acceptable excipient or carrier. As used herein fenretinide includes its derivatives or metabolites. Examples include, but are not limited to, 4-oxo-N-(4-hydroxyphenyl)retinamide (4-oxo-4-HPR) or N-(4-methoxyphenyl)retinamide (4-MPR). As used herein examples of c-KIT inhibitors include, but are not limited to, dasatinib, imatinib, imetelstat, midostaurin, pazopanib, sorafenib and sunitinib or a derivative or salt thereof

As is well known in the art, a pharmaceutically acceptable excipient or carrier is a relatively inert substance that facilitates administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and penetration enhancers. Excipients as well as formulations for parenteral and non-parenteral drug delivery are set forth in Remington's Pharmaceutical Sciences 19th Ed. Mack Publishing (1995).

As used herein “isolated” means a state following one or more purifying steps but does not require absolute purity. “Isolated” extracellular vesicle, exosome or composition thereof means a extracellular vesicle, exosome or composition thereof passed through one or more purifying steps that separate the extracellular vesicle, exosome or composition from other molecules, materials or cellular components found in a mixture or outside of the vesicle, extracellular vesicle or exosome or found as part of the composition prior to purification or separation.

By “effective amount” as used herein with respect to the compositions of the invention, is meant an amount of the extracellular vesicle(s) of the invention, administered to a subject that results in a response by the subject so as to inhibit a disease such as an eye disease, an inflammatory disease, a fibrotic disease such as liver or kidney fibrosis, diabetes, or a cancer. Further, an effective amount may include any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. The particular dosage regimen, i.e., dose, timing and repetition, will depend on the particular individual and that individual's medical history.

As used herein, “treating” means using a therapy to ameliorate a disease or disorder described herein or one or more of the biological manifestations of the disease or disorder; to directly or indirectly interfere with (a) one or more points in the biological cascade that leads to, or is responsible for, the disease or disorder or (b) one or more of the biological manifestations of the disease or disorder; to alleviate one or more of the symptoms, effects or side effects associated with the disease or disorder or one or more of the symptoms or disorder or treatment thereof; or to slow the progression of the disease or disorder or one or more of the biological manifestations of the disease or disorder. Treatment includes eliciting a clinically significant response. Treatment may also include improving quality of life for a subject afflicted with the disease or disorder (e.g., a subject afflicted with a cancer may receive a lower dose of an anti-cancer drug that cause side-effects when the subject is administered with a composition of the invention described herein). Throughout the specification, compositions of the invention and methods for the use thereof are provided and are chosen to provide suitable treatment for subjects in need thereof.

In some embodiments, the methods of treatment described herein is used as a stand-alone therapy without combining with any other therapy.

In other embodiments, the methods of treatment described herein provide adjunct therapy to other therapies, e.g., cancer therapy or any of the diseases described herein, prescribed for a subject. For example, the methods of treatment described herein may be administered in combination with radiotherapy, chemotherapy, gene therapy or surgery. The combination is such that the method of treatment described herein may be administered prior to, with or following adjunct therapy.

In accordance with the invention, the effect of anti-disease or disorder treatment (e.g., a cancer treatment or any of the diseases described herein) may be assessed by monitoring the patient, e.g., by measuring and comparing survival time or time to disease progression (disease-free survival). Any assessment of response may be compared to individuals who did not receive the treatment or were treated with a placebo, or to individuals who received an alternative treatment.

As used herein, “preventing” is understood to refer to the prophylactic administration of a composition of the invention to substantially diminish the likelihood or severity of a condition or biological manifestation thereof, or to delay the onset of such condition or biological manifestation. One skilled in the art will appreciate that prevention is not an absolute term. Prophylactic therapy is appropriate, for example, when a subject is considered at high risk for developing a particular disease or disorder (e.g., a cancer or any of the diseases described herein), such as when a subject has a strong family history of a disease or disorder or when a subject has been exposed to e.g., a disease causing agent, e.g., a carcinogen.

Other than in the operating examples, or where otherwise indicated, all numbers expressing quantities of ingredients or reaction conditions used herein should be understood as modified in all instances by the term “about”. As used herein, the term “about” when used before a numerical designation, e.g., temperature, time, amount, concentration, and such other, including a range, indicates approximations which may vary by (+) or (−) 10%, 5% or 1%.

The use of the singular includes the plural unless specifically stated otherwise. The word “a” or “an” means “at least one” unless specifically stated otherwise. The use of “or” means “and/or” unless stated otherwise. The meaning of the phrase “at least one” is equivalent to the meaning of the phrase “one or more.” Furthermore, the use of the term “including,” as well as other forms, such as “includes” and “included,” is not limiting. The use of the term “containing,” as well as other forms, such as “contains” and “contained,” is not limiting.

### Extracellular Vesicles

The invention provides extracellular vesicle(s) comprising fenretinide. Additionally, the invention provides extracellular vesicle(s) comprising a c-KIT inhibitor.

An extracellular vesicle refers to a membrane that encloses an internal space. Extracellular vesicles may be cell-derived or synthetic bubbles made of the same material as cell membranes, such as phospholipids. Cell-derived extracellular vesicles are smaller than the cell from which they are derived and range in diameter from 20 nm to 5000 nm. Such vesicles can be created through the outward budding and fission from plasma membranes, assembled at and released from a plasma membrane, or derived from cells or vesiculated organelles having undergone apoptosis and may contain organelles. They may be derived from cells by direct and indirect manipulation that may involve the destruction of said cells. They may also be derived from a living or dead organism, explanted tissues or organs, and/or cultured cells. Examples of extracellular vesicles include but are not limited to exosomes, apoptotic bodies, and microvesicles, microparticles, extracellular vesicle-liposome hybrid fusions, and lipid nanoparticles. Cell-derived extracellular vesicles may also include exosomes, ectosomes, shedding vesicles, plasma membrane-derived vesicles and exovesicles. Preferred examples include exosomes, microvesicles and/or apoptotic bodies. A most preferred example of an extracellular vesicle is an exosome.

Exosomes may be secreted membrane-enclosed vesicles that originate from the endosome compartment in cells. The endosome compartment, or the multivesicular body, may be exocytosed from the cell, with ensuing release to the extracellular space of their vesicles as exosomes. Further, an exosome comprises a bilayer membrane, and can comprise various macromolecular cargo either within the internal space, displayed on the external surface of the extracellular vesicle, and/or spanning the membrane. Cargo can comprise nucleic acids, proteins, carbohydrates, lipids, small molecules, and/or combinations thereof. Exosomes may range in size from about 20 nm to 150 mn.

In some embodiments, exosomes and other extracellular vesicles can be characterized and marked based on their protein compositions, such as integrins and tetraspanins. Other protein markers that used to characterize exosomes and other extracellular vesicles include TSG101, ALG-2 interacting protein X (ALIX), flotillin 1, and cell adhesion molecules which are derived from the parent cells in which the exosome and/or EV is formed. Similar to proteins, lipids are major components of exosomes and EVs and can be utilized to characterize them.

Further, naturally occurring exosomes originate from the endosome and may contain proteins such as heat shock proteins (Hsp70 and Hsp90), membrane transport and fusion proteins (GTPases, Annexins and flotillin), tetraspanins (CD9, CD63, CD81, and CD82) and proteins such as CD47. Among these proteins, heat shock proteins, annexins, and proteins of the Rab family are abundantly detected in exosomes and are involved in their intracellular assembly and trafficking. Tetraspanins, a family of transmembrane proteins, are also commonly detected in exosomes. In a cell, tetraspanins mediate fusion, cell migration, cell-cell adhesion, and signaling. Other abundant proteins found in exosomes are the integrins, which are adhesion molecules that facilitate cell binding to the extracellular matrix. Integrins are involved in adhering the vesicles to their target cells. Certain proteins found on the surface of exosomes, such as CD55 and CD59, protect exosomes from lysis by circulating immune cells, while CD47 on exosomes acts as an anti-phagocytic signal that blocks the uptake of exosomes by immune cells. Other proteins associated with exosomes include thrombospondin, lactadherin, ALIX (also known as PDCD6IP), TSG1012, and SDCB 1. Classes of membrane proteins that naturally occur on the surface of exosomes and other extracellular vesicles include ICAMs, MHC Class I, Lamp2b, lactadherin (C1C2 domain), tetraspannins (CD63, CD81, CD82, CD53, and CD37), Tsg101, Rab proteins, integrins, Alix, and lipid raft-associated proteins such as glycosylphosphatidylinositol and flotillin.

Besides proteins, exosomes are also rich in lipids, with different types of exosomes containing different types of lipids. The lipid bilayer of exosomes is mainly constituted of cell plasma membrane types of lipids such as sphingomyelin, phosphatidylcholine, phosphatidylethanolamine, phosphatidylserine, monosialotetrahexosylganglioside (GM3), and phosphatidylinositol. Other types of lipids that can be found in exosomes are cholesterol, ceramide, and phosphoglycerides, along with saturated fatty-acid chains. Additional optional constituents of exosomes include nucleic acids such as micro RNA (miRNA), messenger RNA (mRNA), and non-coding RNAs. They may also contain a sugar (e.g. a simple sugar, polysaccharide, or glycan) or other molecules.

A vesicle preferably has a longest dimension, such as a cross-sectional diameter of at least 10, 20, 30, 40, 50, 60, 70, 80, 90, 100, 200, 300, 400, or 500 nm and/or at most 2000, 1000, 500, 400, 300, 200, 100, 90, 80, 70, 60, or 50 nm. In some embodiments, a longest dimension of a vesicle can range from 10 nm to 1000 nm, 20 nm to 1000 nm, 30 nm to 1000 nm, 10 nm to 100 nm, 20 nm to 100 nm, 30 nm to 100nm, 40 nm to 100 nm, 10 nm to 200 nm, 20 nm to 200 nm, 30 nm to 200 nm, 40 nm to 200 nm, 10 nm to 120 nm, 20 nm to 120 nm, such as 30 nm to 120 nm, 40 nm to 120 nm, 10 nm to 300 nm, nm to 300 nm, 30 nm to 300 nm, 40 nm to 300 nm, 50 nm to 1000 nm, 500 nm to 2000 nm, 100 nm to 500 nm, 500 nm to 1000 nm, and such as 40 nm to 500 nm, each range inclusive. When referring to a plurality of vesicles, such ranges represent the average of all vesicles, including naturally occurring and modified vesicles in the mix.

Merely by way of example, the exosome may have an average range of about 50 nm to about 200 nm in diameter. In an example, exosomes can range in size from about 20 nm to 150 nm. In a preferred embodiment, the exosome has an average diameter of about 120 nm±20 nm.

### Pharmaceutical Compositions

The invention provides provided herein are compositions comprising extracellular vesicles including fenretinide (also referenced herein as “an EV-fenretinide composition of the invention”). Additionally, the invention further provides compositions comprising extracellular vesicles having c-KIT inhibitors (also referred to herein as “an EV-c-KIT composition of the invention”). The compositions maybe in various formulations such as for oral administration, intravenous administration, or intravitreal administration.

Provided herein are compositions comprising an isolated extracellular vesicle(s) comprising fenretinide and a pharmaceutically acceptable excipient. In embodiment, the composition comprises extracellular vesicles having a fenretinide-to-extracellular vesicle ratio which is a value in a range of values between about 0.05 to 30 μg fenretinide per 10⁹ extracellular vesicles or a range of values between about 7.69×10⁴ to 4.61×10⁷ molecules of fenretinide per extracellular vesicle. In some embodiments, the composition may comprise vesicles having about 1.8 ug fenretinide per 10⁹ extracellular vesicles or about 1.82E6 molecules of fenretinide per extracellular vesicle. In some embodiments, the composition may comprise vesicles having a concentration of about 5 e+10 to 4 e+11 vesicles/mL or about 83 pM to 0.66 nM. Other concentrations are contemplated and encompassed by the invention.

In some embodiments, about 95% or more of total fenretinide in the composition is present in or associated with extracellular vesicles. The total fenretinide concentration in one embodiment may be about 6 μM to 30 mM or about 1.4 to 7,000 times above solubility limit of fenretinide in water. In a preferred embodiment, the extracellular vesicle comprises or is an exosome. In a further embodiment, the exosome may be a vesicle with a diameter of about 30 nm to about 150 nm. In some embodiments, less than about 5% of fenretinide in the composition is not associated with an extracellular vesicle.

The composition of the invention may be in the form of a lyophilized powder. In accordance with the invention, the composition may be stable in freezing conditions, e.g., minus 80 degrees Celsius.

In one embodiment, the extracellular vesicle may be obtained from a primary cell culture or a cell line. In a further embodiment, the primary cell culture may be a mesenchymal cell culture, retinal pigment epithelium cell culture, progenitor cell culture, stem cell culture and embryonic stem cell culture. Examples of the cell line include, but are not limited to, HEK293 (kidney epithelial cell line), variants of HEK293 such as HEK293T, FMK 293-F, HEK 293T, and HEK 293-H, dendritic cells, mesenchymal stem cell (MSCs), HT-1080, PER.C6, HeLa, and any variants thereof. Additional optional cells include kidney-specific cell lines such as ACHN (ATCC® CRL-1611™), HK-2 (ATCC® CRL-2190™), A-498 (ATCC® HTB-44™), PK(15) [PK15, PK-15] (ATCC® CCL-33™), Renea (ATCC® CRL-2947™), RPTEC/TERT 1 OCT2 (ATCC® CRL-403 1-OCT2), RPTEC/TERT I OAT3 (ATCC® CRL-403 1-OAT3), RPTEC/TERT I OCT2 (ATCC® CRL-403 1-OCT2), RPTEC/TERT I OATI (ATCC® CRL-4031-OAT I), SV7tert PDGF tumor-I (ATCC® CRL-4008), UMB1949 (ATCC® CRL-4004). RCC cell lines such as 786-0, UM-RC-2, SNU-333, Caski-1, Caski-2, UOK-112, UOK-145, SMKT-R, 769-P, SK-RC-39, SK-RC-42, SK-RC-44, SK-RC-45, SK-RC-46 and variations of these cells. Additional optional cells include CHO-K1 and CHO cell variants such as GS-CHO, and CHO-DG44, Sf9 insect cell line, and NSO and NS I mouse cell lines.

In accordance with the practice of the invention, the composition may be formulated for topical administration. Formulations for topical administration may comprise a cream, foam, gel, balm, lotion, ointment, paste, transdermal skin patch, dermal patch, ear drop, eye drop, vaginal ring, suppository, spray or powder.

Additionally, in some embodiments, the composition may be in a solid dosage form which is a reconstitutable powder. The reconstitutable powder may be placed in a liquid prior to administration.

In other embodiments, the composition may be in a liquid dosage form. For example, such liquid dosage forms may be a suspension or emulsion. The liquid dosage form may be formulated for parenteral administration. Examples of parenteral administration include, but are not limited to, ocular, intraocular, intravitreal, intraosseous, intraperitoneal, intrathecal, intravenous, perivascular, periocular, subconjunctival and transmucosal administration. Additionally, the composition of the invention may also be administered by injection, infusion, lavage, insertion, and implantation.

Further provided herein are compositions comprising an isolated extracellular vesicle(s) comprising a c-KIT inhibitor and a pharmaceutically acceptable excipient. Examples of the c-kit inhibitors may include, but are not limited to, dasatinib, imatinib, imetelstat, midostaurin, pazopanib, sorafenib and sunitinib or a derivative or salt thereof. In some embodiments, the composition comprises vesicles having a concentration of about 1 uM-500 uM. In another embodiment, the composition comprises vesicles having a concentration of about 5 e+10 to 4 e+11 vesicles/mL or about 83 pM to 0.66 nM. In accordance with the practice of the invention, the extracellular vesicle comprises or is an exosome. In some embodiments, less than about 5% of c-KIT inhibitor in the composition is not associated with an extracellular vesicle.

Pharmaceutical compositions disclosed herein may comprise extracellular vesicle(s) of the invention, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions are in one aspect formulated for intravenous administration or intravitreal administration or intranasal administration to the central nervous system.

Pharmaceutical compositions may be administered in a manner appropriate to the disease to be treated (or prevented). The quantity and frequency of administration will be determined by such factors as the condition of the patient, and the type and severity of the patient's disease, although appropriate dosages may be determined by clinical trials.

Suitable pharmaceutically acceptable excipients are well known to a person skilled in the art. The composition can be formulated into a known form suitable for parenteral administration, for example, injection or infusion. The composition may comprise formulation additives such as a suspending agent, a preservative, a stabilizer and/or a dispersant, and a preservation agent for extending a validity term during storage.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, intranasally, intraarterially, intratumorally, into an afferent lymph vessel, by intravenous (i.v.) injection, or intracranially injection, or intraperitoneally (i.p.). In one aspect, the compositions of the present invention are administered to a patient by intraperitoneal injection. In one aspect, the extracellular vesicles compositions described herein are administered by i.v. injection.

### Target Cells

The vesicles described herein can be administered to a cell, tissue, or organ of interest. Preferably, such vesicles are administered to cells of the eye. In some embodiments, the cell of interest is a eukaryotic cell. The cells of interest can be cells from vertebrates such as mammals. Such mammals include primates, including but not limited to, monkeys, chimpanzees, gorillas, and humans. Other examples of vertebrates include birds, such as chickens.

### Production of Vesicles

Delivering fenretinide using EVs can solve the clinical problems of low bioavailability and inability of enough fenretinide to reach its intracellular targets. When incubated under specific conditions, using specific buffers, proteins on the surface of EVs can interact in a distinct manner to stably bind to fenretinide, and package fenretinide on the surface, partially or fully inside the lipid bilayer, or inside the EVs. This can allow a large amount of fenretinide to be delivered on each individual EV. EVs can be naturally taken up by cells and thus introduce fenretinide intracellularly. This can result in more durable treatment responses, more patients treated, and also reduce side effects, such as toxicity, to improve patient quality of life.

In accordance with the invention, methods of making an extracellular vesicle of the invention are provided. In one embodiment, the method comprises the steps of: (a) isolating a vesicle secreted into a culture medium by a producer cell; and (b) introducing fenretinide or c-KIT inhibitors into the vesicle. Suitable examples of producer cells (or cell lines) include, but are not limited to, HEK293 (kidney epithelial cell line), variants of HEK293 such as HEK293T, HEK 293-F, HEK 293T, and HEK 293-H, dendritic cells, mesenchymal stem cell (MSCs), HT-1080, PER.C6, HeLa, and any variants thereof. Additional optional cells include kidney-specific cell lines such as ACHN (ATCC® CRL-1611™), HK-2 (ATCC® CRL-2190™), A-498 (ATCC® HTB-44™), PK(15) [PK15, PK-15] (ATCC® CCL-33™), Renea (ATCC® CRL-2947™), RPTEC/TERT I OCT2 (ATCC® CRL-403 1-OCT2), RPTEC/TERT I OAT3 (ATCC® CRL-403 1-OAT3), RPTEC/TERT I OCT2 (ATCC® CRL-403 1-OCT2), RPTEC/TERT I OATI (ATCC® CRL-4031-0AT I), SV7tert PDGF tumor-I (ATCC® CRL-4008), UMB1949 (ATCC® CRL-4004), RCC cell lines such as 786-0, UM-RC-2, SNU-333, Caski-1, Caski-2, UOK-112, UOK-145, SMKT-R, 769-P, SK-RC-39, SK-RC-42, SK-RC-44, SK-RC-45, SK-RC-46 and variations of these cells. Additional optional cells include CHO-K 1 and CHO cell variants such as GS-CHO, and CHO-DG44, Sf9 insect cell line, and NSO and NS I mouse cell lines.

In some cases, EVs can be cultured via suspension bioreactors using a mesenchymal stem cell (MSC) line. EVs can then be packaged with fenretinide following the exemplary protocol as in Example 1. In some cases, EVs can be cultured via suspension bioreactors using a Chinese Hamster Ovary (CHO) cell line. EVs can then be packaged with fenretinide following the exemplary protocol as in Example 1. In some cases, EVs can be cultured from suspension bioreactors using a cell line, e.g., a HEK293 cell line, and packaged with fenretinide as exemplified in Example 1. Alternatively EVs may be cultured from MSCs adapted to suspension culture, cultured in suspension bioreactors and packaged with fenretinide as in Example 1. In some cases, EVs can be cultured from suspension bioreactors using a HEK293 cell line and packaged with fenretinide as in Example 1. In some cases, they can be stored at −80 C for three weeks until a subject with age-related dry macular degeneration is ready to receive the treatment. The Fenretinide-EV in PBS solution (pH 7.4) can be directly employed for intravitreal or intraocular injection. In some cases, 0.001-0.01 mg/kg dosing level of Fenretinide-EV can exert an intraocular concentration of 2.5E-8 to 5E-6 M to suppress ocular neovascularization (Rebecca Stevens et al., Fenretinide Inhibits Ocular Neovascularization (NV) by Upregulation of Bone Morphogenie Protein-2 (BMP-2) and Reduction of Inflammatory Macrophages and VEGF. Investigative Ophthalmology & Visual Science. 2013. 54, 1956). In some cases, EVs can be cultured from suspension bioreactors using a HEK293 cell line and packaged with fenretinide as in Example 1.

EVs may be stored at about −80 C for a suitable period of time. Merely by way of example, EVs may be stored for about several weeks, e.g. four weeks, until a subject (e.g., a subject with age-related dry macular degeneration, pediatric neuroblastoma or any of the diseases described herein) is ready to receive the treatment.

Additionally, the invention provides methods of preparing and purifying an extracellular vesicle(s) comprising fenretinide or a c-kit inhibitor.

In one embodiment of the invention, the method comprises the step of incubating a suspension of extracellular vesicles (EVs) with a solution comprising fenretinide to prepare an EV-fenretinide formulation. An EV-fenretinide formulation may contain extracellular vesicles comprising fenretinide as described herein infra. The method may further comprise the step of purifying the extracellular vesicles comprising fenretinide in the formulation from fenretinide which is not packaged in an EV.

In another embodiment of the invention, the method includes the step of incubating a suspension of extracellular vesicles (EVs) with a solution comprising a c-kit inhibitor to prepare an EV-c-kit inhibitor formulation. An EV-c-kit inhibitor formulation may contain extracellular vesicles comprising c-kit inhibitor as described herein infra. The method may further comprise the step of purifying the extracellular vesicles comprising c-kit inhibitor in the formulation from c-kit inhibitor which is not packaged in an EV.

In another embodiment of the invention, the method comprises electroporating a suspension of extracellular vesicles (EV) with a solution comprising fenretinide to prepare an EV-fenretinide formulation having extracellular vesicles comprising fenretinide of the invention. The method further comprises the step of purifying the extracellular vesicles comprising fenretinide away from free fenretinide (i.e., fenretinide which is not packaged in an EV).

In an embodiment of the invention, the fenretinide which is not packaged in an EV (free fenretinide) may be a fenretinide aggregate. Alternatively, the fenretinide not packaged in an EV may be soluble extra-vesicular fenretinide (not associated with an EV).

In accordance with the practice of the invention, purifying the extracellular vesicles comprising fenretinide away from free fenretinide may be accomplished through filtration through a membrane filter. Merely by way of example, the membrane filter may be a 0.22-micron membrane filter. Alternatively, purification can involve diafiltration to remove soluble extra-vesicular (not associated with an EV) fenretinide. For example, the diafiltration may be achieved through a centrifugal concentrator. The centrifugal concentrator may use a filter membrane with a molecular cut-off of e.g., about 10 kD to 1,000 kDa. Examples of a centrifugal concentrator include, but are not limited to, Amicon® Ultra-15, Amicon® Ultra-4, Amicon® Ultra-2, and Amicon® Ultra-0.5.

In accordance with the practice of the invention, the extracellular vesicle(s) so purified can include or contain equal to or greater than 95% of the total fenretinide in a sample. Additionally, the extracellular vesicle(s) so purified can comprise less than 5% of total fenretinide as extra-vesicular (not associated with an EV) fenretinide in a sample.

The invention further provides methods of preventing or inhibiting insoluble aggregate formation or precipitation of a fenretinide or a c-KIT inhibitor in an aqueous solution. The method comprises the step of contacting the fenretinide or a c-KIT inhibitor dissolved in a solvent and an extracellular vesicle(s) in an aqueous solution, so as to permit the fenretinide or a c-KIT inhibitor to be incorporated into the extracellular vesicle(s) thereby producing an extracellular vesicle comprising fenretinide or a c-KIT inhibitor of the invention (also referred to herein as an extracellular vesicle of the invention). In this method, the extracellular vesicle(s) may be in an aqueous suspension. Further the fenretinide or a c-KIT inhibitor may have a solubility limit in water of less than 10 microgram/mL or less than 25 μM or a log10 of n-octanal/water partition coefficient (logP) of greater than about 5. Additionally, the fenretinide or a c-KIT inhibitor may form insoluble aggregates or precipitates in the aqueous solution if left in the absence of the extracellular vesicle.

In an embodiment of the invention, the extracellular vesicle(s) of the invention comprises more than 10-fold over the solubility limit of the fenretinide or a c-KIT inhibitor in water. In another embodiment, wherein the extracellular vesicle(s) of the invention comprises more than 100-fold over the solubility limit of the fenretinide or a c-KIT inhibitor in water. In yet another embodiment, the extracellular vesicle(s) of the invention comprises more than 1,000-fold over the solubility limit of the fenretinide or a c-KIT inhibitor in water. In a further embodiment, the extracellular vesicle(s) of the invention comprises more than 7,000-fold over the solubility limit of the fenretinide or a c-KIT inhibitor in water.

In accordance with the invention, the extracellular vesicle(s) of the invention in the aqueous solution may be further fractionated or purified to remove fenretinide or a c-KIT inhibitor not incorporated into an extracellular vesicle. Optionally, the method further comprises the step of changing the buffer of the aqueous solution following fractionation or purification of the extracellular vesicle(s) of the invention. In accordance with the practice of the invention, the extracellular vesicle(s) so purified can include or contain equal to or greater than 95% of the total fenretinide or c-KIT inhibitor in a sample. Additionally, the extracellular vesicle(s) so purified can comprise less than 5% of total fenretinide or c-KIT inhibitor as extra-vesicular (not associated with an EV) fenretinide or c-KIT inhibitor in a sample. Alternatively, the extracellular vesicle(s) of the invention comprises 99% or greater of total fenretinide or c-KIT inhibitor after fractionation or purification. In a specific embodiment, the extracellular vesicle(s) of the invention comprises 99.5% or greater of total fenretinide or c-KIT inhibitor after fractionation or purification.

In one example, the extracellular vesicle(s) of the invention is in a composition and the extracellular vesicle(s) of the invention comprises 98% or greater of total fenretinide or c-KIT inhibitor in the composition after fractionation or purification. In another example, less than 2% of total fenretinide or c-KIT inhibitor in the composition may be found outside of the extracellular vesicle(s) of the invention in the composition. In yet another embodiment, less than 1% of fenretinide or c-KIT inhibitor in the composition may be found outside of the extracellular vesicle(s) of the invention in the composition.

The invention additionally provides a method of preparing an aqueous suspension of a concentrated fenretinide or c-KIT inhibitor beyond its solubility limit in water. The method comprises the step of contacting an extracellular vesicle(s) as an aqueous suspension and fenretinide or c-KIT inhibitor dissolved in a solvent so as to permit incorporation of the fenretinide or c-KIT inhibitor in an extracellular vesicle and produce an extracellular vesicle(s) of the invention. Further, the method provides the step of purifying extracellular vesicle(s) of the invention in the aqueous suspension away from extravesicular fenretinide or c-KIT inhibitor. Then, the method provides an optional step of concentrating and/or changing buffer of the extracellular vesicle(s) of the invention so as to further increase the concentration of the fenretinide or c-KIT inhibitor as a suspension and/or change the buffer of the aqueous suspension. In accordance with this method, the fenretinide or c-KIT inhibitor may have a solubility limit in water of less than 10 microgram/mL or less than 25 μM or a log10 of n-octanal/water partition coefficient (logP) of greater than 5. The purified and optionally concentrated extracellular vesicle(s) of the invention and/or changed buffer has an fenretinide or c-KIT inhibitor concentration above the solubility limit of fenretinide or c-KIT inhibitor in water. Further, fenretinide or c-KIT inhibitor concentration achieved following incorporation into extracellular vesicles may exceed the solubility limit of the fenretinide or c-KIT inhibitor alone in an aqueous solution.

In an embodiment of the invention, a c-kit inhibitor may be hydrophilic and soluble in water with a solubility limit of at least 10 mg/mL, preferably above 100 mg/mL. Merely by way of example, the hydrophilic c-kit inhibitor may include, but are not limited to, dasatinib hydrochloride (water solubility limit of 42 mg/mL; see Selleck Chem Catalog No. S5254; CAS No. 854001-07-3) and imatinib mesylate (water solubility limit of 118 mg/mL; see Selleck Chem Catalog No. S1026; CAS No. 220127-57-1). In accordance with the practice of the invention, the hydrophilic c-kit inhibitor may be incorporated into extracellular vesicle by indirect bath sonication, active loading or electroporation. Merely by way of example, methods for incorporating a hydrophilic c-kit inhibitor may include any of the methods as described in Example 17, e.g., for incorporating dasatinib hydrochloride or imatinib mesylate. Additionally, the hydrophilic c-kit inhibitor which incorporated into an extracellular vesicle may be purified or fractionated from unincorporated free c-kit inhibitor using one or more of the protocols as described in Example 17. Further, the extracellular vesicle comprising a hydrophilic c-kit inhibitor may have or comprise a drug concentration selected from any value between about 2.5 μM and 454 μM or any value as provided in Table 4. In one embodiment of the invention, the extracellular vesicle comprising the hydrophilic c-kit inhibitor may be produced from a suspension of extracellular vesicles and a hydrophilic c-kit inhibitor with an encapsulation efficiency of at least about 1%, 2%, 3%, 4%, 5%, 6%, 7%, or 8% or any encapsulation efficiency between about 0.03% and 8.7% or any value as provided in Table 4. Further still, an extracellular vesicle comprising a hydrophilic c-kit inhibitor may comprise a particle concentration anywhere between 4E7 and 8E10 or any of the values as provided in Table 4. In a further embodiment, the extracellular vesicle comprising a hydrophilic c-kit inhibitor may comprise an average median particle size anywhere between 70 nm diameter and 160 nm or any of the average median particle size as provided in Table 4. In yet a further embodiment, the extracellular vesicle comprising a hydrophilic c-kit inhibitor may comprise an average median particle size anywhere between about 70 nm diameter and 160 nm and a percent median standard error with a value falling between about 0.4% and 52%. In still a further embodiment, the extracellular vesicle comprising a hydrophilic c-kit inhibitor may include any of the average median particle size and percent median standard error as provided in Table 4. Further still, the extracellular vesicle comprising a hydrophilic c-kit inhibitor may comprise drug molecules per extracellular vesicle having a value anywhere between about 3E4 and 1E10 or any of the drug molecules per extracellular vesicle as provided in Table 4. In another embodiment, the extracellular vesicle comprising a hydrophilic c-kit inhibitor comprises a combination of final drug concentration, percent final drug packaging/encapsulation efficiency, final particle concentration, average median particle size with percent median standard error and drug molecules per extracellular vesicle as provided in Table 4. In an embodiment, the drug molecule is or comprises dasatinib hydrochloride. In a different embodiment, the drug molecule is or comprises imatinib mesylate. In an embodiment, the hydrophilic c-kit inhibitor is dasatinib hydrochloride. In a different embodiment, the hydrophilic c-kit inhibitor is imatinib mesylate.

In an embodiment, c-kit inhibitor may be hydrophobic and be insoluble in water (e.g., dasatinib (see Selleck Chem Catalog No. S1021; CAS No. 302962-49-8) or imatinib (see Selleck Chem Catalog No. S2475; CAS No. 152459-95-5)). Examples of the hydrophobic c-kit inhibitor may include, but are not limited to, dasatinib and imatinib. In an embodiment, the hydrophobic c-kit inhibitor is incorporated into extracellular vesicle by incubation or sonication. In an embodiment, the hydrophobic c-kit inhibitor is incorporated into extracellular vesicle direct probe sonication. In an embodiment, the method of incorporating the hydrophobic c-kit inhibitor is any of the methods as provided in Example 17 for dasatinib or imatinib. In an embodiment, the hydrophobic c-kit inhibitor incorporated into an extracellular vesicle is purified or fractionated from unincorporated free c-kit inhibitor using one or more of the protocols as described in Example 17. In an embodiment, the extracellular vesicle comprising the hydrophopbic c-kit inhibitor has or comprises a drug concentration selected from any value between 0.5 μM and 45 μM or any value as provided in Table 3. In an embodiment, the extracellular vesicle comprising the hydrophilic c-kit inhibitor is produced from a suspension of extracellular vesicles and a hydrophilic c-kit inhibitor with an encapsulation efficiency of at least 0.2%, 1%, 2%, 3%, 4%, 5%, 6% or 7%, or any encapsulation efficiency between 0.2% and 7.0% or any value as provided in Table 3. In an embodiment, the extracellular vesicle comprising a hydrophobic c-kit inhibitor comprises a particle concentration anywhere between 4E10 and 1.2E11 or any of the values as provided in Table 3. In an embodiment, the extracellular vesicle comprising a hydrophobic c-kit inhibitor comprises an average median particle size anywhere between 125 nm diameter and 140 nm or any of the average median particle size along as provided in Table 3. In an embodiment, the extracellular vesicle comprising a hydrophobic c-kit inhibitor comprises an average median particle size anywhere between 125 nm diameter and 140 nm and percent median standard error selected anywhere between 0.2% and 1.7%. In an embodiment, the extracellular vesicle comprising a hydrophobic c-kit inhibitor comprises any of the average median particle size along with percent median standard error as provided in Table 3. In an embodiment, the extracellular vesicle comprising a hydrophobic c-kit inhibitor comprises drug molecules per extracellular vesicle having a value anywhere between 3E3 and 1.1E6 or any of the drug molecules per extracellular vesicle as provided in Table 3. In an embodiment, the extracellular vesicle comprising a hydrophilic c-kit inhibitor comprises a combination of final drug concentration, percent final drug packaging/encapsulation efficiency, final particle concentration, average median particle size with percent median standard error and drug molecules per extracellular vesicle as provided in Table 3. In an embodiment, the drug molecule is or comprises dasatinib. In a different embodiment, the drug molecule is or comprises imatinib. In an embodiment, the hydrophilic c-kit inhibitor is dasatinib. In a different embodiment, the hydrophilic c-kit inhibitor is imatinib

In an embodiment, c-kit inhibitor is incorporated into extracellular vesicle by any of the method described in this application, wherein the c-kit inhibitor may be any of dasatinib, imatinib, imetelstat, midostaurin, pazopanib, sorafenib and sunitinib or a derivative or salt thereof. In an embodiment, the extracellular vesicle of the invention may be an extracellular vesicle comprising a c-kit inhibitor selected from the group consisting of dasatinib, imatinib, imetelstat, midostaurin, pazopanib, sorafenib and sunitinib or a derivative or salt thereof.

In an embodiment, the composition comprising an isolated extracellular vesicle(s) comprising a c-KIT inhibitor and a pharmaceutically acceptable excipient comprises a c-kit inhibitor concentration anywhere between 10 μM-500 μM.

Further provided is an extracellular vesicle made, purified or isolated by any of the production methods of the invention as described herein.

The extracellular vesicles so made can be incorporated with the fenretinide or a c-KIT inhibitor directly with or without cholesterol or other phospholipids. The extracellular vesicle protein mixture can be created via gentle mixing and incubation or several cycles of freezing and thawing.

The vesicles can be derived from eukaryotic cells that can be obtained from a subject (autologous) or from allogeneic cell lines. The vesicles can be obtained from a subject, from primary cell culture cells obtained from a subject, from cell lines (e.g., immortalized cell lines), and other cell sources. The subject may be any living organisms, vertebrates or non-vertebrates. Examples of subjects include humans, plants, mouse, rat, hamster, guinea pig, cat, dog, monkey, hamster, pig, donkey, chicken, goat, cow, sheep, or horse, and transgenic species thereof. In a preferred embodiment, the vertebrate is a primate. Examples of the primate include. but are not limited to, a human, old world monkey, new world monkey, chimpanzee, gorilla, orangutan, gibbon, tarsier, lemur, loris and allies.

Vesicles can be concentrated and separated from the circulatory cells using centrifugation, filtration, or affinity chromatography columns. In some embodiments, the vesicles are derived from skeletal muscle tissue and can be concentrated and separated using centrifugation, filtration or affinity chromatography columns.

As described above, the extracellular vesicle(s) of the invention can also be associated with or fused with other delivery vehicles, such as liposomes or adeno-associated viral vectors to enhance delivery to target cell. See György, Bence, et al. Biomaterials 35 (2014)26:7598-7609.

Additionally, the fenretinide or c-KIT inhibitor in the vesicle(s) can be combined with another payload, for example, a small molecule, polypeptide, nucleic acid, lipid, carbohydrate, ligand, receptor, reporter, drug, or combination of the foregoing (e.g., two or more drugs, or one or more drugs combined with a lipid, etc.). Examples of payloads, include, for example therapeutic biologics (e.g., antibodies, recombinant proteins, or monoclonal antibodies), RNA (siRNA, shRNA, miRNA, antisense RNA, mRNA, noncoding RNA, tRNA, rRNA, other RNAs), reporters, lipids, carbohydrates, nucleic acid constructs (e.g., viral vectors, plasmids, lentivirus, expression constructs, other constructs), oligonucleotides (e.g., including synthetic antisense oligonucleotides), aptamers, cytotoxic agents, anti-inflammatory agents, antigenic peptides, small molecules, and nucleic acids and polypeptides for gene therapy. Payloads can also be complex molecular structures such as viral nucleic acid constructs (encoding transgenes) with accessory proteins for delivery to target cells where the nucleic acid construct can be (if needed) reverse transcribed, delivered to the nucleus, and integrated (or maintained extrachromosomally). Payloads may be loaded into the extracellular vesicle internal membrane space, displayed on, or partially or fully embedded in the lipid bi-layer surface of the extracellular vesicle. Examples of pharmaceutical and biologic payloads include drugs for treating diseases (such as organ diseases), conditions and syndromes, cytotoxic agents, and anti-inflammatory drugs. Examples of the disease or condition is selected from the group consisting of liver fibrosis, lung fibrosis, renal fibrosis, glaucoma, ocular hypertension, leukoplakia, diabetes, obesity, insulin resistance and hepatic steatosis.

Examples of RNA payloads include siRNAs, miRNAs, shRNA, antisense RNAs, small nuclear RNA (snRNA), small nucleolar RNA (snoRNA), long intergenic noncoding RNA (lincRNA), piwi interacting RNA (piRNA), ribosomal RNA (rRNA), tRNA, and rRNA. Examples of noncoding RNA payloads include microRNA (miRNA), long non-coding RNA (lncRNA), small nuclear RNA (snRNA), small nucleolar RNA (snoRNA), long intergenic non-coding RNA (lincRNA), piwi-interacting RNA (piRNA), ribosomal RNA (rRNA), yRNA, circular RNA (circRNA) and transfer RNA (tRNA). miRNAs and lncRNAs in particular are powerful regulators of homeostasis and cell signaling pathways, and delivery of such RNAs by an EV can impact the target cell.

Reporters are moieties capable of being detected indirectly or directly. Reporters include, without limitation, a chromophore, a fluorophore, a fluorescent protein, a luminescent protein, a receptor, a hapten, an enzyme, and a radioisotope.

Examples of reporters include one or more of a fluorescent reporter, a bioluminescent reporter, an enzyme, and an ion channel. Examples of fluorescent reporters include, for example, green fluorescent protein from Aequorca victoria or Renilla reniformis, and active variants thereof (e.g., blue fluorescent protein, yellow fluorescent protein, cyan fluorescent protein, etc.); fluorescent proteins from Hydroid jellyfishes, Copepod, Ctenophora, Anthrozoas, and Entacmaea quadricolor, and active variants thereof; and phycobiliproteins and active variants thereof. Chemiluminescent reporters include, for example, placental alkaline phosphatase (PLAP) and secreted placental alkaline phosphatase (SEAP) based on small molecule substrates such as CPSD (Disodium 3-(4-methoxyspiro {1,2-dioxetane-3,2′-(5′-chloro)tricyclo [3.3.1.13,7]decan}-4-yl)phenyl phosphate, β-galactosidase based on 1,2-dioxetane substrates, neuraminidase based on NA-Star® substrate, all of which are commercially available from ThermoFisher Scientific. Bioluminescent reporters include, for example, aequorin (and other Ca+2 regulated photoproteins), luciferase based on luciferin substrate, luciferase based on Coelenterazine substrate (e.g., Renilla, Gaussia, and Metridina), and luciferase from Cypridina, and active variants thereof. In some embodiments, the bioluminescent reporter include, for example, North American firefly luciferase, Japanese firefly luciferase, Italian firefly luciferase, East European firefly luciferase, Pennsylvania firefly luciferase, Click beetle luciferase, railroad worm luciferase, Renilla luciferase, Gaussia luciferase, Cypridina luciferase, Metrida luciferase, OLuc, and red firefly luciferase, all of which are commercially available from ThermoFisher Scientific and/or Promega. Enzyme reporters include, for example, β-galactosidase, chloramphenicol acetyltransferase, horseradish peroxidase, alkaline phosphatase, acetylcholinesterase, and catalase. Ion channel reporters include, for example, cAMP activated cation channels. The reporter or reporters may also include a Positron Emission Tomography (PET) reporter, a Single Photon Emission Computed Tomography (SPECT) reporter, a photoacoustic reporter, an X-ray reporter, and an ultrasound reporter.

Nucleic acid payloads can be oligonucleotides, recombinant polynucleotides, DNA, RNA, or otherwise synthetic nucleic acids. The nucleic acids can cause splice switching of RNAs in the target cell, turn off aberrant gene expression in the target cell, replace aberrant (mutated) genes in the chromosome of the target cell with genes encoding a desired sequence. The replacement nucleic acids can be an entire transgene or can be short segments of the mutated/aberrant gene that replaces the mutated sequence with a desired sequence (e.g., a wild-type sequence). Alternatively, the nucleic acid payloads can alter a wild-type gene sequence in the target cell to a desired sequence to produce a desired result. The payload nucleic acids can also introduce a transgene into the target cell that is not normally expressed. The payload nucleic acids can also cause desired deletions of nucleic acids from the genome of the target cell.

Appropriate genome editing systems can be used with the payload nucleic acids such as CRISPR, TALEN, or Zinc-Finger nucleases. The efficiency of homologous and non-homologous recombination can be facilitated by genome editing technologies that introduce targeted double-stranded breaks (DSB). Examples of DSB-generating technologies are CRISPR/Cas9, TALEN, Zinc-Finger Nuclease, or equivalent systems. See, e.g., Cong et al. Science 339.6121 (2013): 819-823, Li et al. Nucl. Acids Res (2011): gkr188, Gajet al. Trends in Biotechnology 31.7 (2013): 397-405, all of which are incorporated by reference in their entirety for all purposes. Payload nucleic acids can be integrated into desired sites in the genome (e.g., to repair or replace nucleic acids in the chromosome of the target cell), or transgenes can be integrated at desired sites in the genome including, for example, genomic safe harbor site, such as, for example, the CCRS, AAVS1, human ROSA26, or PSIP1 loci. Sadelain et al., Nature Rev. 12:51-58 (2012); Fadel et al., J. Virol. 88(17):9704-9717 (2014); Ye et al., PNAS 111(26):9591-9596 (2014), all of which are incorporated by reference in their entirety for all purposes. When a CRISPR system is used, Cas9 in the target cell may be derived from a plasmid encoding Cas9, an exogenous mRNA encoding Cas9, or recombinant Cas9 polypeptide alone or in a ribonucleoprotein complex. Kim et al (2014) Genome 1012-19. doi:10.1101/gr.171322.113; Wang et al (2013) Cell 153 (4). Elsevier Inc.: 910-18. doi:10.1016/j.ce11.2013.04.025, which are incorporated by reference in their entirety for all purposes.

BLAST 2 Sequences is another tool that can be used for comparing protein and nucleotide sequences (see FEMS Microbiol Lett. 1999 174(2): 247-50; FEMS Microbiol Lett. 1999 177(1): 187-8 and the website of the National Center for Biotechnology information at the website of the National Institutes for Health).

### 1. Introducing Fenretinide or c-KIT Inhibitor

Fenretinide or a c-KIT inhibitor may be incorporated into vesicles through several methods involving physical manipulation. Physical manipulation methods include but are not limited to, electroporation, sonication, mechanical vibration, extrusion through porous membranes, electric current and combinations thereof, which cause disruption of vesicle membrane. Loading of fenretinide or c-KIT inhibitor to vesicles described herein may involve passive loading processes such as mixing, co-incubation, or active loading processes such as electroporation, sonication, mechanical vibration, extrusion through porous membranes, electric current and combinations thereof. In some embodiments, said loading can be done concomitantly with vesicle assembly.

Fenretinide or c-KIT inhibitors can be passively loaded into vesicles by incubation to allow diffusion into the vesicles along the concentration gradient. The hydrophobicity of the drug molecules can affect the loading efficiency. Hydrophobic drugs can interact with the lipid layers of the vesicle membrane and enable stable packaging of the drug in the vesicle's lipid bilayer. In some embodiments, purified exosome solution suspended in buffer solution can be incubated with payload. In some preferred embodiments, the fenretinide or c-KIT inhibitor is dissolved in a solvent mixture that can include DMSO, to allow passive diffusion into exosomes. Following this, the payload-exosomes mixture is made free from un-encapsulated payload. In preferred embodiments, centrifugation or size-exclusion columns are used to remove precipitates from the supernatant. LC/MS methods can be used for the measurement and characterization of payload in the exosome-payload formulation, following lysis and removal of the exosome fraction.

Fenretinide or c-KIT inhibitors can be diffused into cells by incubation with cells that then produce exosomes that carry those substances. For example, cells treated with a fenretinide or c-KIT inhibitor can secrete exosomes loaded with them (Pascucci, L. et. al, Journal of Controlled Release, 192 (2014): 262-270).

Extracellular vesicles secreted from cells can be mixed with fenretinide or c-KIT inhibitors and subsequently sonicated by using a homogenizer probe. The mechanical shear force from the sonicator probe can compromise the membrane integrity of the exosomes and subsequently allow the drug to diffuse into the exosomes during this membrane deformation.

In another embodiment, extracellular vesicles from cells can be mixed with a fenretinide or c-KIT inhibitors, and the mixture can be loaded into a syringe-based lipid extruder with 100-400 nm porous membranes under a controlled temperature. The exosome membrane can be disrupted during the extrusion process can allow vigorous mixing with the drug. In some examples, the number of effective extrusions can vary from 1-10 to effectively deliver drugs into exosomes.

Fenretinide or c-KIT inhibitors can be incubated with exosomes at room temperature for a fixed amount of time. Repeated freeze-thaw cycles are then performed to ensure drug encapsulation. The method can result in a broad distribution of size ranges for the resulting exosomes, and then, the mixture is rapidly frozen at −80° C. or in liquid nitrogen and thawed at room temperature. The number of effective freeze-thaw cycles may vary from 2-7 for effective encapsulation. In another embodiment, membrane fusion between exosomes and liposomes can be initiated through freeze-thaw cycles to create exosome-mimetic particles.

In another cases, small pores can be created in exosomes membrane through application of an electrical field to exosomes suspended in a conductive solution. The phospholipid bilayer of the exosomes can be disturbed by the electrical current. Fenretinide or c-KIT inhibitors can subsequently diffuse into the interior of the exosomes via the pores. Thintegrity of the exosome membrane can then be recovered after the drug loading process. In some examples, siRNA or miRNA can be loaded into exosomes using this method.

In some cases, electroporation can be conducted in an optimized buffer such as trehalose disaccharide to aid in maintaining structural integrity and can inhibit the aggregation of exosomes.

Membrane permeabilization can be initiated through incubation with surfactants such as saponin. In some examples, hydrophilic molecules can be assisted in exosome encapsulation by this process.

Chemistry based approaches can also be used to directly attach molecules to the surfaces of exosomes via covalent bonds. In some examples, copper-catalyzed azide alkyne cycloaddition can be used for the bioconjugation of small molecules and macromolecules to the surfaces of exosomes as shown in Wang et. al, 2015 and Hood et. al, 2016 [Here the references are incorporated in their entirety].

In another embodiment, fluorophores and microbeads conjugated to highly specific antibodies can bind a particular antigen on the cell surface. Specific antigen-conjugated microbeads can be used for exosome isolation and tracking in vivo.

Fenretinide or c-KIT inhibitors carried by the extracellular vesicle(s) can include, for example, miR-133a (downregulate miR-133a targets Smarcd1 and Runx2), other miRNA can include miR-1, miR-133, miR-133b, miR-181a-5p, miR-206, and miR-499.

In some cases, EV samples in the range of 5 e+10 to 4 e+11 particles/ml are prepared in different buffers (e.g., PBS). As described herein, fenretinide can be dissolved in DMSO solution. Merely by way of example, the Fenretinide/100% DMSO stock solution can be in the range of 2.5 mM to 50 mM, e.g., in the range of 5 mM to 10 mM. EV samples can be diluted with PBS depending upon the desired concentration of EVs (about 5+10 or 4 e+11 particles/ml) and an appropriate amount of 10 mM fenretinide stock is added to achieve the desired drug mass (μg) to 1E9 EV particle count ratio. The ratios can range from 0.05 to 30, e.g., 0.5 to 2. The final DMSO concentration in the Fenretinide-EV sample can range from 0.002% to 25%, e.g., 0.5 to 10%. The EVs can be mixed with the Fenretinide/100% DMSO stock solution by pipetting up and down, e.g., a couple of times, and proceeding to the desired incubation conditions. Incubation temperatures can range from room temperature, 24° C., to 37° C. or up to 45° C. Incubation times can range from about <1 minute to 16 hours or in some cases, up to about 24 hours.

After drug has been incubated with the EV sample, the Fenretinide-EV samples can be transferred to different filter and filtered. Examples of such filter include, but are not limited to, 0.22 μm SpinX filters (˜500 μL), 0.22 μm Steriflip filters (˜50 mL), or 0.22 μm Stericup filters (˜150 mL to 1 L). Filtration may be performed by centrifugation or by syringe filtration. In some cases, centrifugation is performed at about 1000 g. In some cases, the sample is transferred to a syringe attached to a 0.22 μM Millex filter and the Fenretinide-EV solution is filtered. The filtered material can be collected and transferred directly to an Amicon-15 concentrator tube with molecular weight cutoff (MWCO) ranging from about 10-100 kDa. In some cases, the centrifugal filter tube is pre-cleaned by adding PBS and spinning at about 3000 g for about 10 min. The flow-through is removed. In some cases, a buffer exchange with PBS is performed by adding PBS to the Amicon centrifugal filtration tube. The filtered Fenretinide/EV solution can be added to the Amicon centrifugal filtration tube. The tube can be spun at about 3000 g for 15 minutes or longer and flow through is removed. Next, PBS can be added to the Amicon centrifugal filtration tube and mixed well by pipetting. The Amicon centrifugal filtration tube can be filled with PBS. The tube can be then spun at 3000 g for 15 minutes or longer to remove the flow through. This step can be repeated from 1 to 5 times, by adding PBS to the Amicon centrifugal filtration tube, mixing well by pipetting, and centrifugating to remove the flow through. The Amicon centrifugal filtration tube can then be filled with PBS. The tube can be spun at 3000 g for 15 minutes or longer to remove the flow through.

The cleaned up Fenretinide-EV sample can be further filtered and then centrifuged to achieve a sterile, clean mixture, free of aggregates and non-packaged drug. Merely by way of example, filtration can involve a 0.22 μm Spin-X filter or Millex filter and centrifugation can involve about 1000 g for 5 minutes.

The final drug amount, concentration and packaging efficiency may be quantified from the final packaged Fenretinide-EV samples using well known techniques. For example, the amount of drug present can be quantified using absorption spectra measured using a plate reader. In some cases, the final Fenretinide-EV sample is diluted 10 times in PBS. About 200 μl of the sample may be loaded into the UV-transparent plate or read directly on the plate reader. In order to measure the concentration of the Fenretinide in the Fenretinide-EV samples, a standard curve for the drug concentration may be generated. Merely by way of example, a standard curve may be generated as follows: a serial dilution (2×) is performed of the Fenretinide/100% DMSO stock in DMSO/PBS 50:50 solution. A serial dilution can be used to create a concentration range from 0.5 μM to 125 μM. Direct addition of Fenretinide/100% DMSO stock to PBS leads to precipitate and fails to obtain accurate standard curve. The fenretinide-DMSO samples can also be loaded at 200 μl in the wells. The absorption spectra can be measured by the plate reader (about 250 to 800 nm range). The linear fit from the standard curve built from the Fenretinide/DMSO/PBS samples can be used to calculate the concentration of Fenretinide in the Fenretinide-EV samples. In addition, Fenretinide-EV samples can be quantified by HPLC methods or LC-MS methods. In addition, samples may be run on the NTA Nanosight for checking size (particle diameter in nm) and particle concentration (particles/ml).

### Uses of Organ/Tissue/Cell Specific EVs

Exosomes or exosome mimetics have many of the desirable features of an ideal drug delivery system, such as a long circulating half-life, the intrinsic ability to target tissues, biocompatibility, and minimal or no inherent toxicity issues. Alternatively, liposomes or polymeric nanoparticles can be modified with extracellular vesicle(s) of the invention to make modified liposomes or polymeric nanoparticles that can traffic to desired locations, interact with target cells, and fuse with target cells to deliver fenretinide or c-KIT inhibitors.

The invention provides methods of treating a disease or condition in a subject in need thereof by administering an extracellular vesicle(s) of the invention or compositions thereof into the subject in a sufficient amount so as to treat the subject. Preferably, the extracellular vesicles are derived from eukaryotic cells that can be obtained from a subject (autologous) or from allogeneic cells (or cell lines). HLA compatibility is preferred in the case of allogenic cells or cell lines. However, the extracellular vesicles can be derived from non-autologous or non-allogeneic subject or cells.

Examples of such diseases or conditions include, but are not limited to, liver fibrosis, lung fibrosis, renal fibrosis, glaucoma, ocular hypertension, leukoplakia, diabetes, obesity, insulin resistance and hepatic steatosis. The disease can also include tumors or cancers such as non-small cell lung cancer, T-cell lymphoma, cutaneous T-cell lymphoma, unclassified cutaneous T-cell lymphoma, peripheral T-cell lymphoma, not classified peripheral T-cell .lymphoma, primary cutaneous T-cell lymphoma, follicular T-cell lymphoma, angioimmunoblastic T-cell lymphoma and Sézary's disease. The vesicle(s) of the invention can treat eye diseases. Examples of eye diseases or conditions include: corneal and anterior segment diseases, retinoblastoma, uveal melanoma, retinoid pigmentosa, diabetic retinopathy, diabetic macular edema, Type 1 macular telangiectasia, Type 2 macular telangiectasia, Stargardt disease, uveitis, scleritis, and retinopathy of prematurity.

Merely by way of example, a fenretinide-EV composition (also referred to herein as an EV-fenretinide formulation or fenretinide-EV formulation) in PBS solution (about pH 7.4) may be directly employed for subconjunctival injections without an addition of the organic solvent (e.g., DMSO). About 5 uL of 5-150 uM of Fenretinide-EV formulation can be administered daily subconjunctivally to treat fungal keratitis (Zhao W, et al., Fenretinide Inhibits Neutrophil Recruitment and IL-1β Production in Aspergillus fumigatus Keratitis. Cornea, 2018, 37(12):1579-1585)). In some cases, a composition of the invention (e.g. fenretinide-EV in PBS) (pH 7.4) can be added with viscosity enhancer (e.g., polyvinyl alcohol (PVA)) (David S. Jones. Topical drug dosage forms for eye conditions. The Pharmaceutical Journal, P J June 2017; Takuya Fujisawa', Hiroko Miyai, Kohei Hironaka, Toshimasa Tsukamoto, Kohei Tahara, Yuichi Tozuka, Masakilto, and Hirofumi Takeuchi. Liposomal diclofenac eye drop formulations targeting the retina: Formulation stability improvement using surface modification of liposomes. International Journal of Pharmaceutics. 2012, Pages 564-567) and preservatives, for multiple usage, to form into eye drop formulation for ocular topical administration. Merely by way of example, one drop of the about 5-150 uM of Fenretinide-EV eye drop formulation may be administered once or four times daily to treat anterior uveitis or any of the eye diseases described herein).

In some cases, extracellular vesicle(s) of the invention can be used as treatment for patients with existing cancer in cancer types such as breast cancer (e.g., estrogen receptor positive breast cancer), prostate cancer, general brain cancers, pediatric neuroblastoma, ovarian cancer, renal cell carcinoma, non-small cell lung cancer, general solid tumors, hematologic malignancies, and glioblastomas.

Specifically, the invention provides a method of treating cancer comprising administering to a subject in need thereof an effective amount of an EV-fenretinide composition of the invention so as to treat cancer in the subject.

Further provided herein is a method of treating an eye disease comprising administering to a subject in need thereof an effective amount of an EV-fenretinide composition of the invention so as to treat the eye disease in the subject. Examples of the eye disease include, but are not limited to, retinoblastoma, uveal melanoma, retinoid pigmentosa, diabetic retinopathy, diabetic macular edema, Type 1 macular telangiectasia, Type 2 macular telangiectasia, macular degeneration (e.g., wet age-related macular degeneration, dry age-related macular degeneration, and Stargardt disease), uveitis, scleritis, and retinopathy of prematurity.

Specifically, the invention provides a method of inhibiting macular degeneration comprising administering to a subject in need thereof an effective amount of an EV-fenretinide composition of the invention so as to inhibit angiogenesis, vascular leakage, or loss of retinal pigment epithelium; or restores retinal pigment epithelium or restores retina or promotes transdifferentiation of retinal pigment epithelium to neuronal cell type thereby inhibiting macular degeneration in the subject. In some embodiments, the macular degeneration may be dry age-related macular degeneration. Examples of dry age-related macular degeneration include any of drusen or geographic atrophy. In other embodiments, the macular degeneration may be wet age-related macular degeneration. In additional embodiments, the macular degeneration may be Stargardt disease, juvenile macular degeneration or fundus flavimaculatus.

In some embodiments, an EV-fenretinide composition of the invention accumulates preferentially at the lens and provides a reservoir of fenretinide intraocularly. The composition may be administered intravenously or intravitreally. In one embodiment, intravitreal administration is intravitreal injection.

Another method provided herein is a method of treating cystic fibrosis comprising administering to a subject in need thereof an effective amount of an EV-fenretinide composition of the invention so as to treat the cystic fibrosis in the subject. Also provided herein is a method of treating rheumatoid arthritis comprising administering to a subject in need thereof an effective amount of an EV-fenretinide composition of the invention so as to treat the rheumatoid arthritis in the subject.

Also, the invention provides methods of selectively modulating retinoic acid receptor activity in an eye of a subject. In one embodiment, the method comprises administering an effective amount of an EV-fenretinide composition of the invention to the eye of the subject. The method further comprises permitting an eye cell in the eye of the subject to contact the vesicles in the composition for a sufficient period so that the vesicles in the composition fuse and/or are internalized by the eye cell and fenretinide is released intracellularly. In yet another embodiment, the method further comprises allowing the fenretinide so released to bind a retinoic acid receptor of the eye cell, so as to modulate activity of the retinoic acid receptor. The invention further provides a method for treating an eye disease in a subject by selectively modulating retinoic acid receptor activity in the eye of the subject by a method of the invention.

Additionally, the invention provides a method for selectively increasing fenretinide concentration in an eye of a subject comprising administering an EV-fenretinide composition of the invention to the eye of the subject so as to increase ocular or intraocular concentration of fenretinide without a comparable increase in concentration outside of the eye, thereby selectively increasing fenretinide concentration in the eye of the subject. The invention further provides a method for treating an eye disease in a subject by selectively increasing fenretinide concentration in an eye of the subject by a method of the invention. In some embodiments, administration comprises topical administration or intraocular administration. Topical administration may comprise or is administration of topical eye drops. Intraocular administration may comprise or is intravitreal administration. Intravitreal administration may comprise or is intravitreal injection.

In some embodiments, the eye disease involves the cornea of a subject and the EV-fenretinide composition of the invention so administered involves delivery of an effective amount of fenretinide to the cornea and/or to conjunctiva of the eye. The eye disease may be keratitis or corneal ulcer. In one embodiment, the keratitis is or comprises fungal keratitis. Examples of the fungal keratitis may include, but are not limited to, Aspergillus fumigatus keratitis and Fusarium solani keratitis. In some embodiments, the subject infected in the eye with Aspergillus fumigatus or Fusarium solani keratitis is at risk of visual impairment and blindness due to the infection. In some embodiments, the subject in need is at risk of loss of corneal clarity.

In some embodiments, the effective amount of fenretinide suppresses inflammation at the cornea. In some embodiments, the effective amount of fenretinide reduces neutrophil recruitment and inflammatory cytokine production at the cornea.

For example, a composition of the invention (e.g. fenretinide-EV in PBS) may be employed for intravenous injection formulation directly without the use of any organic solvent (e.g., 10% ethanol in saline (Isabella Orienti, et al., A new bioavailable fenretinide formulation with antiproliferative, antimetabolic, and cytotoxic effects on solid tumors. Cell Death & Disease; 2019; 10: 529). About 1-10 mL/kg dosing volume, 0.1-5 mg/kg dosing level of Fenretinide-EV in PBS may be administered every day or every other day via intravenous injection to treat tumors. In some cases, the about 250-1000 mg/mm2 Fenretinide-EV formulation can be dosed daily for the first 5 days, every 21 days to exert antitumor activity in peripheral T-cell lymphomas. In some cases, the Fenretinide-EV formulation of the invention can be prepared in aqueous solution (e.g., PBS) for oral formulation. In some cases, the EV-fenretinide formulation can then be lyophilized (freeze dried) and administered as capsules to produce an oral formulation (Jitinder S. Wilkhu, et al., Development of a solid dosage platform for the oral delivery of bilayer vesicles. Eur J Pharm Sci. 2017; 108: 71-77). About 0.5-30 mg/kg/week dosing level of oral Fenretinide-EV formulation can be administered to treat tumors. In some cases, 100-1800 mg/m2 of Fenretinide-EV capsules or liquid formulations can be administered orally, divided twice or thrice daily, 7 days every three weeks to stabilize ovarian cancer [Reynolds CP et al., High plasma levels of fenretinide (4-HPR) were associated with improved outcome in a phase II study of recurrent ovarian cancer: A study by the California Cancer Consortium [abstract]. Proc Am Soc Clin Oncol 2007; 25: 5555-5555].

Specifically, the invention provides a method of treating a tumor or cancer comprising administering to a subject in need thereof an effective amount of an EV-c-KIT inhibitor composition of the invention so as to treat the cancer or tumor in the subject. The tumor or cancer may be an eye cancer, uveal melanoma, retinoblastoma, GIST/gastrointestinal cancer, pancreatic cancer, liver cancer, mastocytosis tumor, triple-negative breast cancer, non-small cell lung cancer, small cell lung cancer, leukemia and renal cell carcinoma. Also provided herein is a method of treating dry age-related macular degeneration in a subject comprising administering to the subject in need thereof an effective amount of an EV-c-KIT composition of the invention so as to inhibit progression of dry AMD or restore RPE of the eye, thereby, treating dry age-related macular degeneration of the subject. Further provided herein is a method of treating diabetes-induced hyperpermeable retinal vasculature in a subject comprising administering to the subject in need thereof an effective amount of an EV-c-KIT composition of the invention so as to inhibit retinal vascular leakage in an eye of the subject, thereby, treating diabetes-induced hyperpermeable retinal vasculature in the subject.

The desired amount of fenretinide or c-KIT inhibitor on an EV may consider the target cell concentration, density of markers on the target cell, whether target cells are associated with other target cells, target cells' local microenvironment, the binding affinity (K_{d}) of fenretinide or c-KIT inhibitor for a marker on the target cell, and the concentration of delivery vehicle.

Packaging fenretinide or c-KIT inhibitors with EVs can solve the problem of delivering a highly hydrophobic drug like fenretinide or hydrophobic c-KIT inhibitors (e.g., dasatinib and imatinib) or hydrophilic c-KIT inhibitors in salt form (e.g., Dasatinib Hydrochloride or Imatinib Mesylate.

### Kits

According to another aspect of the invention, kits are provided. Kits according to the invention include package(s) comprising the extracellular vesicle(s) or compositions of the invention. In one embodiment, the kit comprises the extracellular vesicle(s) of the invention and instructions for use and/or storage.

The phrase “package” means any vessel containing compounds or compositions presented herein. In preferred embodiments, the package can be a box or wrapping. Packaging materials for use in packaging pharmaceutical products are well known to those of skill in the art. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment.

The kit can also contain items that are not contained within the package but are attached to the outside of the package, for example, pipettes.

Kits may optionally contain instructions for administering vesicles or compositions of the present invention to a subject having a condition in need of treatment. Kits may also comprise instructions for approved uses of compounds herein by regulatory agencies, such as the United States Food and Drug Administration. Kits may optionally contain labeling or product inserts for the present vesicles or compositions of the invention. The package(s) and/or any product insert(s) may themselves be approved by regulatory agencies. The kits can include active agents in the solid phase or in a liquid phase (such as buffers provided) in a package. The kits also can include buffers for preparing solutions for conducting the methods, and pipettes for transferring liquids from one container to another.

The kit may optionally also contain one or more other compositions for use in combination therapies as described herein. In certain embodiments, the package(s) is a container for intravenous administration. In other embodiments, compounds are provided in an injectable means.

The inventions disclosed herein will be better understood from the experimental details which follow. However, one skilled in the art will readily appreciate that the specific methods and results discussed are merely illustrative of the inventions as described more fully in the claims which follow thereafter. Unless otherwise indicated, the disclosure is not limited to specific procedures, materials, or the like, as such may vary. It is also to be understood that the terminology used herein is for the purpose of describing particular embodiments only and is not intended to be limiting.

### EXAMPLE 1: Production of EVs and Procedure for EV-fenretinide Packaging

EVs were produced from cell culture using a standardized procedure. Briefly, HEK293 cells were cultured in suspension, seeded at 1E7 (1×10⁷) cells/mL in chemically-defined growth medium, in a shaking flask with a shaking speed of 125 rpm, and incubated at 37° C. and 8% CO₂ atmosphere in a shaking-platform incubator (Infors Multitron). 24 hours after seeding, the culture was harvested, cells were removed by centrifugation at 130×g for 5-10 minutes, and the supernatant collected. The supernatant was further processed by centrifugation at about 3,200×g for 5 minutes, followed by sterile filtration with a 0.22 μm PES filter. Sterile clarified harvest fluid was stored at −80° C. prior to EV isolation. EVs were isolated and purified from thawed, clarified harvest fluid at room temperature or 37° C. by first concentrating the clarified harvest fluid using a 100 kDa MWCO membrane (either by Amicon® Ultra diafiltration concentrators (Sigma/Millipore) or TFF systems, e.g., Pall tangential flow filtration (TFF) system), and then performing diafiltration into 1× PBS. The EVs are retained on the membrane. The concentrated, buffer-exchanged preparation is then further purified with CaptoCore 700 mixed mode resin (Cytiva) to remove soluble proteins from the solution, and the final EV sample is filter-sterilized with a 0.22 μm PES filter prior to use or storage.

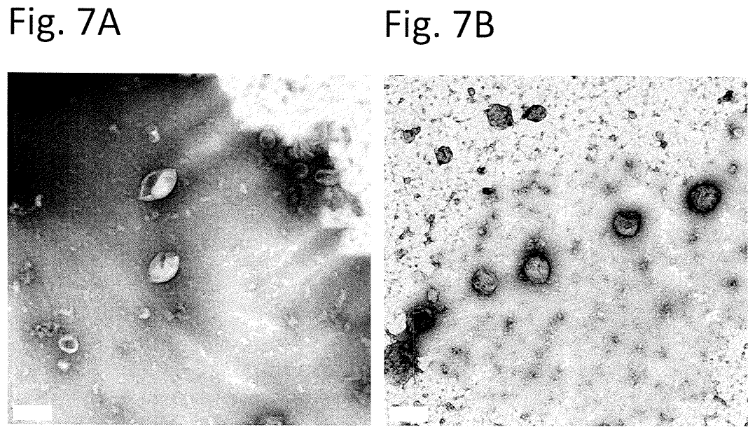

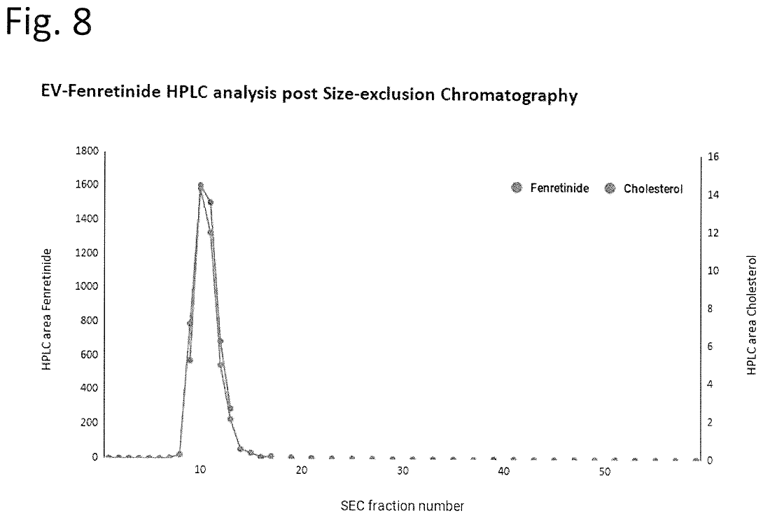

The isolated EV samples can have a diameter size of 50-220 nm (e.g., with a median diameter size range of about 120 nm-170 nm) with a concentration of about 1E10 (1×10¹⁰) to 1E12 (1×10¹²) particles per mL (e.g., about 7E10 to 2E11 particles per mL) as measured by Nanoparticle Tracking Analysis, NTA, (e.g., NanoSight NS300, Malvern). The EV samples can have a diameter size distribution of about 80-200 nm and a particle concentration of 1E8 to 1E10 particles per mL as measured by vesicle flow cytometry (Cytoflex, Beckman Coulter). The EV samples can have a protein concentration of about 100 to 10,000 μg/mL, or about 500-700 μg/mL as measured by Qubit 4 fluorometer (ThermoScientific). The EV samples have an impurity metric of 1-13 or 2-6 μg of proteins per 1E9 particles. The EV samples have tetraspanins (e.g., CD81, CD9, and CD62) on the EV surface as measured by vesicle flow cytometry (Cytoflex, Beckman Coulter) or ELISA. About 10-80%, or about 30-40% of the EVs have CD81 proteins, about 1-80% or about 5-20% of the EVs have CD9 proteins, about 0.1-30%, or about 1-15% of the EVs have CD63 proteins. Alternatively, in an embodiment, an EV population may be obtained to comprise anywhere between 1-99% of a particular EV marker, such as, for example CD81, CD9, CD63 or any protein found on EVs, using for example, a fractionation or purification setup comprising an AKTA flux S TFF system (GE healthcare). The EV samples comprise EV-related proteins such as Rab9, TSG101, and AnnexinV and non-EV specific proteins such as GAPDH and Actin as measured by Western blot (WES, ProteinSimple). The lipid composition of the EV samples contains cholesterol, as shown in Example 11, FIG. 8. The EV samples appear cup-shaped particles under transmission electron microscopy (TEM) images in Example 10, FIG. 7A.

An EV-fenretinide was packaged using the protocol provided below:

This protocol allowed packaging of 118.2 μg of drug inside EV (2.0E11 particles) with a 24.9% encapsulation efficiency and a final concentration of 604 μM Fenretinide-EV in PBS solution. In this protocol, Amicon® Ultra-15 diafiltration concentrators were used to produce the batch sizes necessary for in vivo animal studies. In another embodiment, similar protocols could be developed for small batch sizes using smaller Amicon® Ultra centrifugation filters (Sigma/Millipore), such as Amicon® Ultra-4 and Amicon® Ultra-0.5.

HEK293 cell derived EVs were prepared in the PBS buffer at a concentration of 2.10E+11 particles/mL. Fenretinide (Selleck Chemicals: Cat No: 55233) was dissolved in 100% DMSO. The fenretinide/DMSO stock solution can be 5 mM or 10 mM.

Protocol for introducing fenretinide into EV to obtain EV comprising fenretinide (fenretinide-EV, also called fen-EV, EV-fen or EV-fenretinide) and purifying the fenretinide-EV from extra-vesicular (not associated with an EV) fenretinide not incorporated into EVs:

A. Exosome/drug substance incubation:
1. The EV samples were diluted with PBS to a final EV concentration around 2.10E11 particles per mL.
2. For every 2,000 μL of exosome solution, 128.67 μL of 10 mM fenretinide in 100% DMSO were added to achieve a drug substance (fenretinide) mass (in μg) to 1E9 EV count ratio of 1.2 in the appropriate tube format. (Final DMSO concentration amount for this sample was ˜6% of the total volume.)
3. The EVs with the drug solution were mixed by pipetting up and down a couple of times at room temperature, with no extended incubation, then processed immediately according to the clean-up steps below.

B. Corning® Costar® SpinX® (0.22 μm cellulose acetate membrane filter) centrifuge tube filter for large drug substance aggregates removal
1. 2,000 μL of fenretinide-EV samples were split into four 0.5 mL SpinX® filters (Corning® Costar®, VWR, 29442-752; 0.22 μm cellulose acetate membrane filter) and filtered by centrifugation at 1,000×g.
2. Samples were pooled to obtain ˜2 mL volume and aliquots were collected pre- and post-filtration for in-process analysis.

C. Amicon Ultra-15 centrifugal filter for removing free/non-packaged drug substance:
1. 5 mL of 1× PBS buffer was added to rinse the 100 kDa Amicon® Ultra-15 concentrator tube (Sigma/Millipore, UFC910024) and centrifuged at 3,220×g for 5 mins. The flow through was removed.
2. (Wash 1) 13 mL of 1× PBS was added to each concentrator tube. 2 mL of filtered fenretinide-EV sample was added to the concentrator tube and centrifuged at 3,220×g until the sample was concentrated to ˜0.5 mL. The flow through was discarded.
3. (Wash 2) 14 mL of PBS was added to each concentrator tube and mixed well by pipetting. The tube was centrifuged at 3,220×g until sample and was concentrated to ˜0.5 mL. The flow through was discarded.
4. (Wash 3) 14 mL of PBS was added to each concentrator tube and mixed well by pipetting. The tube was centrifuged at 3,220×g until sample and was concentrated to ˜0.5 mL. The flow through was discarded.
5. P200 Pipetman® was used to rinse the concentrator membrane of each tube with the concentrated sample. Fenretinide-EV samples were transferred to a 0.22 μm Spin-X filter, filtered by centrifuging at 1,000×g for 5 minutes to obtain a final sterilized sample.

E. Post-packaging QC metrics:
1. Drug quantitation: Final drug substance amount, concentration and packaging were quantified from the final drug packaged EV sample. Final drug quantitation was performed using absorption spectra from the plate reader (using Synergy H1M, BioTek) using the following steps:
   a. The final fenretinide-EV sample was diluted 10' in PBS, and 200 μL were loaded into the UV-transparent plate (Corning, cat no 3635)
   b. The standard curve was generated by performing 2× serial dilutions (from about 125 μM to 0.5 μM) of the fenretinide/100% DMSO stock in DMSO/PBS (50:50) solution to generate a standard curve. The fenretinide/DMSO/PBS standard curve samples also had 200 μL in the wells.
   c. The absorption spectra were measured (250 to 800 nm range) by a BioTek Synergy H1 plate reader.
   d. The linear fit from the standard curve generated from the fenretinide/DMSO/PBS samples was used to interpolate the concentration of fenretinide in the fenretinide-EV samples.
2. Size measurements: Samples were run on Nanoparticle Tracking Analysis (NTA) NanoSight NS300 (Malvern) to determine EV particle size distribution and count using a sample volume ranging from about 1 μL to 1,000 μL.

### EXAMPLE 2: Packing of Fenretinide in EVs Using Double Distilled Water (ddH2O) and Clean Up by Microdialysis Device for High Throughput Screening

A. Exosome buffer exchange (using Amicon® Ultra-4 centrifugal filter):
1. 2 mL of pooled exosome samples in PBS with a particle concentration of 4.3E11 particles/mL were added into 100 kDa Amicon® Ultra-4 tube (Sigma/Millipore)
2. The tube was centrifuged at 3,000×g for 5 mins. The flow through was removed. The collection was around 2 mL.
3. 2 mL of double distilled water (ddH₂O) was added to the tube and spun at 3,000×g for 10 mins. The flow through was removed. The collection was around 500 μL.
4. 3 mL of ddH₂O was added to the tube and spun at 3,000×g for 10 mins. The flow through was removed. The collection was around 500 μL.
5. 3 mL of ddH₂O was added to the tube and spun at 3,000×g for 7 mins. The flow through was removed. The collection was around 250 μL.
6. 1.5 mL of the ddH₂O was added to the collection tube. P200 Pipetman® was used to retrieve the solution (˜2 mL).
7. Nanoparticle Tracking Analysis (NTA) measurement was performed on NanoSight NS300 (Malvern) to determine EV particle size distribution and count using a sample volume ranging from about 1 μL to 1,000 μL.

B. Exosome/drug incubation:
1. 150 μL of the 2E11/mL EV samples was incubated with 15 μL of 5 mM fenretinide in 100% DMSO in plastic Eppendorf tubes at 37° C. for 18 hr.

C. Corning® Costar SpinX® (0.22 μm cellulose acetate membrane filter) centrifuge tube filter clean up:
1. 150 μL of the incubated fenretinide-EV samples was transferred to each 0.22 μm SpinX® filter tube (Corning® Costar®, VWR 29442-752) and spun at 14.000×g for 10 mins. The supernatant was collected for micro-dialysis device clean up, below.

D. Micro-dialysis device clean up:
1. Add 1,500 μL of the ddH₂O into a row of the deep well plate associated with the microanalysis kit (Pierce 96-well Microdialysis plate, 10K MWCO. ThermoFisher Scientific, 88260).
2. Add 100 μL of the ddH₂O to the cassette to wet the dialysis membrane.
3. Gently place the micro-dialysis device into the deep well plate with ddH₂O.
4. Gently pipet the ddH₂O out by tuning the pipet to 140 μL.
5. Pipet the 100 μL of the supernatant (i.e., fenretinide-EV with removal of large aggregates) to each pre-wet micro-dialysis filter cassette and incubated at RT for 3 hr.
6. Remove the existing dialysis buffer (i.e., ddH2O) in each well and exchange with 1.5 mL of new ddH2O and incubated at 4° C. overnight.
7. Remove the existing dialysis buffer (i.e., ddH2O) in each well and exchange with 1.5 mL of new ddH₂O and incubated at RT for 3 hrs.
8. Remove the existing dialysis buffer (i.e., ddH2O) in each well and exchange with 1.5 mL of new ddH2O and incubated at 4° C. overnight.

E. Fenretinide-EV quantitation and characterization was similar to “post processing QC metric” of Example 1
9. Absorption spectra measurement was performed on a Spectramax M2 microplate reader (Molecular Devices, San Jose, Calif.). following the manufacturer's suggestion using 100 μL of sample volume.
10. Nanoparticle Tracking Analysis (NTA) measurement of particle size and concentration was performed using a sample volume ranging from about 1 μL to 1000 μL.

The resulting EV-fenretinide formulation was then evaluated in terms of efficiency of free fenretinide clean-up and fenretinide encapsulation. Free fenretinide (i.e., fenretinide not incorporated into an EV or fenretinide free of an EV) clean-up is an important metric because free fenretinide tends to aggregate due to its high hydrophobicity. Fenretinide was detected and quantified based on absorption spectra on plate reader and LC MS/MS. EV-fenretinide and negative control samples were run in triplicates and results were averaged. The negative control (also run in triplicates) was 15 μL of 5 mM fenretinide/100% DMSO stock added to 150 μL of ddH₂O (note negative control lacks EVs but other is the same fenretinide concentration and solution condition used to package fenretinide into EV), subjected to the entire clean-up protocol. Following clean-up, fenretinide concentration in negative controls was near or below the limit of detection, ensuring confidence that the fenretinide measured in the EV-fenretinide formulation is truly packaged with the EVs and not remaining as free fenretinide. Three positive controls of fenretinide without the packaging/cleanup process were also measured to ensure proper calibration of the LC MS/MS machine. The success of EV-fenretinide packaging was measured using the following metrics of clean-up efficiency calculation and drug-encapsulation efficiency calculation.

Clean up efficiency calculation:

This calculation provides a measure of amount of fenretinide not removed by the clean-up protocol.

[1−(negative control after clean-up fenretinide concentration/starting fenretinide concentration)]×100%

Note that the starting fenretinide concentration for the negative control is the same as the concentration of fenretinide used to produce EVs comprising fenretinide. Further, the incubation condition is the same except in one case EVs are present (associated with) and in the other (the negative control) no EV is present. Also, note that the starting fenretinide concentration is also the concentration used to prepare the positive control samples, which were not subjected to packaging and clean-up procedures.

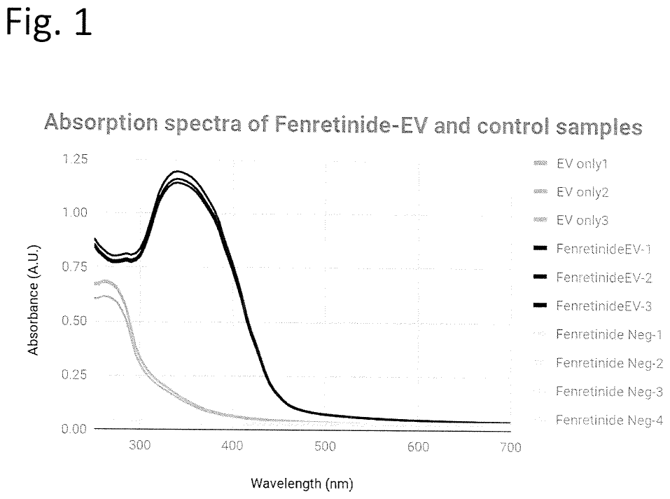

FIG. 1 shows EV-fenretinide absorption spectra as compared to fenretinide-alone samples and EV-alone samples for Example 2. Each entity has clear, distinctive peak. The negative control (non-EV packaged fenretinide), subjected to the entire packaging and drug cleanup protocol showed no distinct fenretinide peak at 375 nm, ensuring confidence to the fenretinide clean-up process.

Drug encapsulation efficiency calculation:

This calculation quantifies how much drug is actually packaged with the EVs, compared to how much fenretinide was input as starting material at the beginning of a drug packaging protocol.

$$\frac{\begin{matrix}Drug concentration of E V - \\ drug formulation measured at end of entire protocol , including cleanup\end{matrix}}{Starting drug loading concentration ( positive control )} \times 100 \%$$

TABLE 1
Results for Example 2, averaged across the three EV-fenretinide
replicates. Negative controls were below the Limit of Detection
(LOD). Columns denoted with “plate reader” were quantities
measured using absorption spectra on a plate reader. Columns
denoted with “LC-MS” were quantities measured using
a LC MS/MS mass spectrometry system. A detectable encapsulation
efficiency was observed, meaning there was a measurable amount
of fenretinide being packaged with the EVs.

| Plate Reader | Plate Reader | | LC-MS |
|---|---|---|---|
| clean up | encapsulation | LC-MS | encapsulation |
| efficiency | efficiency | concentration | efficiency |
| 99.89% | 3.62% (at 415 nm) | 28.84 uM | 3.36% |
| | | (negative | |
| | | controls: <LOD = | |
| | | 0.0125 uM) | |

### Example 3: Packing of Fenretinide in EVs Using a Tris Buffer

EVs were produced from HEK293 cell culture, harvested from hollow fiber bioreactors, and isolated using size exclusion chromatography (SEC) columns. Fenretinide-EV packaging was performed using the protocol listed in Example 1, except using a Tris-based buffer. 500 μL of EV samples with 9.55E10 particles in 20 mM Tris. about pH 6.5 were mixed with 5 mM Fenretinide stock in 100% DMSO to reach a final fenretinide concentration of 711 μM and 14% DMSO (vol/vol). Exactly the same amount (82.9 μL) of 5 mM Fenretinide stock in 100% DMSO was added to 500 μL, 1×PBS solution as fenretinide-alone control. Exactly the same amount (82.9 μL) of DMSO was added to 500 μL EV sample to reach 14% DMSO (vol/vol) as EV-alone control. All three samples (fenretinide-EV, fenretinide-alone control, and EV-alone control) were subjected to the same clean-up process as described in Example 1.

Fenretinide in EV-fenretinide, fenretinide-alone control, and EV-alone control samples were quantified via plate reader absorption spectra.

### Example 4: Measurement of Fenretinide-EV Ratio

EVs are produced from HEK293 cell culture, harvested from hollow fiber bioreactors, and isolated using SEC columns. EV-Fenretinide packaging was performed using water in one sample batch, and in the second sample batch, EV-fenretinide packaging was produced using Tris as an incubation buffer.

The effect of varying fenretinide concentration on its incorporation into EVs was determined by holding the concentration of EVs constant (1.2E10 particles in 100 μL ddH₂O or 6.59E9 particles in 100 μL 20 mM Tris buffer, pH 6.5) and incubating the EVs with different concentrations of fenretinide (500 μM to 5 mM using a 50 mM fenretinide stock in 100% DMSO or 15-250 μM using a 1 mM fenretinide stock in 100% DMSO). Fenretinide packaging and clean-up protocol as described in Example 2 was followed. The final purified fenretinide-EV product obtained after the clean-up protocol was analyzed for the amount of fenretinide present in or on the EVs (associated with the EVs) and normalized for EVs based on absorption spectrum of the purified fenretinide-EV product using a plate reader. Normalization was performed by taking the absorbance measured at 375 nm and dividing by the absorbance measured at 260 nm, the former reflecting the amount of fenretinide in the sample and the latter reflecting the amount of EV in the same sample.

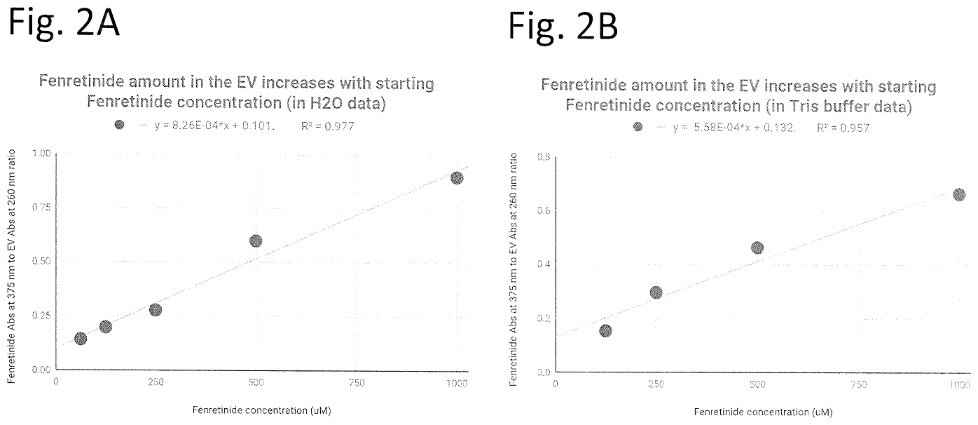

FIG. 2 shows plots a ratio of absorbance measured at 375 nm over absorbance measured at 260 nm (y-axis) as a function of the starting fenretinide concentration used to prepare fenretinide-EVs (x-axis) in ddH₂O (FIG. 2A) or a 20 mM Tris buffer, pH 6.5 (FIG. 2B). Linear regression analysis shows positive linear correlation with a positive correlation coefficient of greater than 0.95 (r²), indicating that contacting a fixed amount or concentration of EVs with an increasing amount or concentration of fenretinide leads to increasing amount of fenretinide incorporated into EVs, showing that EVs have a large capacity as delivery vehicle or carrier for fenretinide. In addition, EV as a delivery vehicle or carrier can carry a wide range of doses of fenretinide. By increasing the amount of starting drug concentration and holding the amount of EVs constant, a linear increase in the end drug-to-EV ratio (packaged drug/number EVs counted) was observed.

### Example 5: MCF-7 Cell Viability with EV-Fenretinde Dosing

EV-fenretinide was packaged in a manner similar to the protocol described in Example 2. EV-fenretinide was dosed onto MCF-7 breast cancer cells using the exemplary protocol below. Cell viability was measured using the Promega CellTiter-Glo® Luminescent Cell Viability Assay—a method of determining the number of viable cells in culture based on quantitation of the ATP present, an indicator of metabolically active cells.

Protocol steps:
1. MCF-7 cells were cultured in Dulbecco's Modified Eagle's Medium (DMEM) supplemented with 10% exosome-free fetal bovine serum (FBS) supplemented with penicillin-streptomycin-glutamine (PSG; 100 units/mL penicillin, 100 μg/mL streptomycin and 2 mM L-glutamine) for 1-2 passages.
2. MCF-7 cells were harvested, washed, counted and resuspended in culture medium (DMEM supplemented with 10% exosome-free FBS and PSG) at 5E5 cells/mL.
3. The MCF-7 cells were seeded at 5E4 cells in a volume of 100 μL/well of culture medium in a white polystyrene 96-well tissue culture plate and incubated for 18-20 hrs at 37° C./5% CO₂ humidified incubator.
4. After 18-20 hrs of incubation, medium was removed from the wells and replaced with culture medium containing test agents. Test agents were prepared in culture medium by serial two-fold dilutions to generate an 8-point curve and 100 μL of the prepared test agents were placed into each well. Test agent samples were:
   a. Fenretinide alone formulation in 100% DMSO (Positive control)—concentration range 40, 20, 10, 5, 2.5, 1.25, 0.625, 0 μM fenretinide;
   b. Fenretinide-EV formulation in PBS—concentration range 40, 20, 10, 5, 2.5, 1.25, 0.625, 0 μM fenretinide;
   c. EV alone—EV concentrations present in fenretinide-EV formulation in PBS test agent sample (in “b,” above) are matched in the EV alone test agent samples at 4.06E9, 2.03E9, 1.02E9, 5.08E8, 2.54E8, 1.27E8, 6.34E7, 0 EV particles per 100 μL.
   d. DMSO (Vehicle for Fenretinide) alone—concentration range of DMSO alone is matched to concentration of fenretinide alone formulation test agent sample (above) with DMSO concentration (vol/vol) of 0.8%, 0.4%, 0.2%, 0.1%, 0.05%, 0.025%, 0.0125% and 0%.
   e. The total volume for each well was 100 μL and all were set up in triplicate.
5. The plate(s) was incubated at 37° C./5% CO₂ humidified incubator for 48 hours.
6. After exposure to test agents, the plated cells and Cell Titer-Glo (Promega G7570) reagents were brought to room temperature to equilibrate for 30 minutes.
7. 100 μL of the Cell Titer-Glo® reagent was added to each well. The plate was shaken for two minutes and then left to equilibrate for 10 minutes prior to reading the luminescence on a microplate reader (iD3-Molecular Devices).

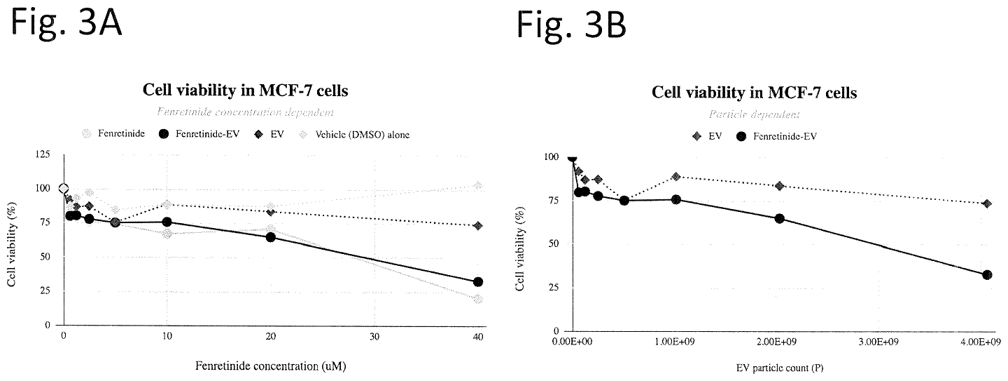

FIG. 3 shows a graph of percent cell viability based on relative luminescence units (RLU) as a function of fenretinide concentration as well as EV particle count. In addition, FIG. 3A shows treatment with EV alone or vehicle (DMSO) alone corresponding to matched amount of EVs in fenretinide-EV test agent sample and fenretinide alone test agent sample, respectively. Fenretinide and fenretinide-EV test agents both displayed a dose-dependent cell viability inhibition, most notable after about 10 μM of fenretinide concentration. Cell viability was about 20% at 40 μM fenretinide for the Fenretinide alone test agent sample and 32% at 40 μM fenretinide for the Fenretinide-EV test agent sample, while even at the highest concentration for the controls matched for EV concentration in fenretinide-EV test agent sample (namely, the EV alone test agent sample), cell viability is less affected with a viability of about 73%. In the case of DMSO alone test agent sample, no drop in cell viability is observed at the highest DMSO concentration tested in the DMSO alone teat agent sample corresponding to the DMSO concentration in the 40 μM fenretinide of the fenretinide alone test agent sample (see FIG. 3A). Thus, cancer cells, such as human breast cancer cells (e.g., MCF-7) are sensitive to treatment with fenretinide either packaged as in fenretinide-EV test agent or dissolved in DMSO as in fenretinide alone test agent. Loss in cell viability is more prominent the higher the dose of fenretinide.

FIG. 3B shows when analyzed for the amount of EV particles used to treat MCF-7 human breast cancer cells, the EV devoid of fenretinide (EV alone test agent sample) appears to have slight effect on viability of MCF-7 cells, decreasing by about 25% at the highest EV concentration of 4.06E9/100 μL reaching a cell viability of about 75% of the level of untreated sample. The reason and significance for this slight drop in cell viability (based on quantitation of ATP present in metabolically active cells) is not clear. With the introduction of fenretinide into EV as in fenretinide-EV, the same EV concentration (4.06E9 EV/100 μL) with a concentration of fenretinide at 40 μM results in a significant drop in viability from about 75% viability for EV-alone to about 32% viability for fenretinide-EV test agent. The additional drop in cell viability by about 43 percentage point is consistent with previous reports of fenretinide as an anti-neoplastic agent and apoptotic agent toward cancer cells, especially at higher doses. Thus, fenretinide incorporated into EV is bioavailable and active, and fenretinide-EV is an effective agent for inhibiting growth of cancer cells, including human cancer cells.

### EXAMPLE 6: Time-Dependent Concentration Measurement in Blood and Tissues of Mice Injected with EV-Fenretinide Formulation

Fenretinide-alone was formulated with excipients 5% Ethanol, 12.5% Solutol-HS 15, 12.5% PEG 300, and 70% PBS and then tested in increasing doses in CD-1® IGS (Charles River) male mice. During formulation testing of the free fenretinide and excipients, an adverse event was observed in one mouse, which stopped moving for several minutes after an injection of 6.6 mg fenretinide-alone formulation/kg mouse. This highlighted the potential risks of free fenretinide formulations, and how EV-fenretinide could allow for injections of potentially higher doses.

EVs were produced and harvested from HEK293 cell culture using suspension shake flask bioreactors. They were isolated using SEC gel filtration columns. EVs were then packaged with fenretinide using the exemplary protocol provided in Example 1. This exemplary protocol resulted in a packaging efficiency of 24%, the highest achieved packaging efficiency using incubation-based methods. Two mice arms with nine 6-8 week-old CD-1 male mice in each arm and approximately 25 grams per mouse assessed the effect of intravenous injection of either fenretinide with excipients 5% Ethanol, 12.5% Solutol-HS 15, 12.5% PEG 300, and 70% PBS (Arm 1) or EV-fenretinide in PBS (Arm 2). The batch of EV-fenretinide injected into mice was characterized and were shown for a total volume of 1,000 μL to have 128 μg of fenretinide, 8.26E10 particles/mL, 189 μg/mL of total protein counts as measured by Qubit, and 13.1 ng/μL total DNA counts.

Both arms were dosed via intravenous tail-vein injected at 0.23 mg/kg with a 4 mL/kg dose volume. The EV-fenretinide formulation was stored at −80° C. until injection into mice. Blood was collected at 2, 5, 10, 15, 30, 60, 120, 240, and 960 minutes, three mice were sacrificed at each time point, and tissue was collected at 30, 120 and 960 minutes. Spleen, liver, lung, kidney, brain and heart tissues were collected. Fenretinide amount in blood and tissue was measured via LC MS/MS mass spectrometry. Serum and hematology toxicology measurements were collected from the blood samples. Blood urea nitrogen (BUN), creatinine, and alanine aminotransferase were measured from serum.

Toxicology measurements were compared against industry standard baselines for mouse studies. Both fenretinide and EV-fenretinide formulations did not exert apparent adverse effects based on serum and blood chemistry results, consistent with the notion that there was no additional toxicity contributed from the EVs.

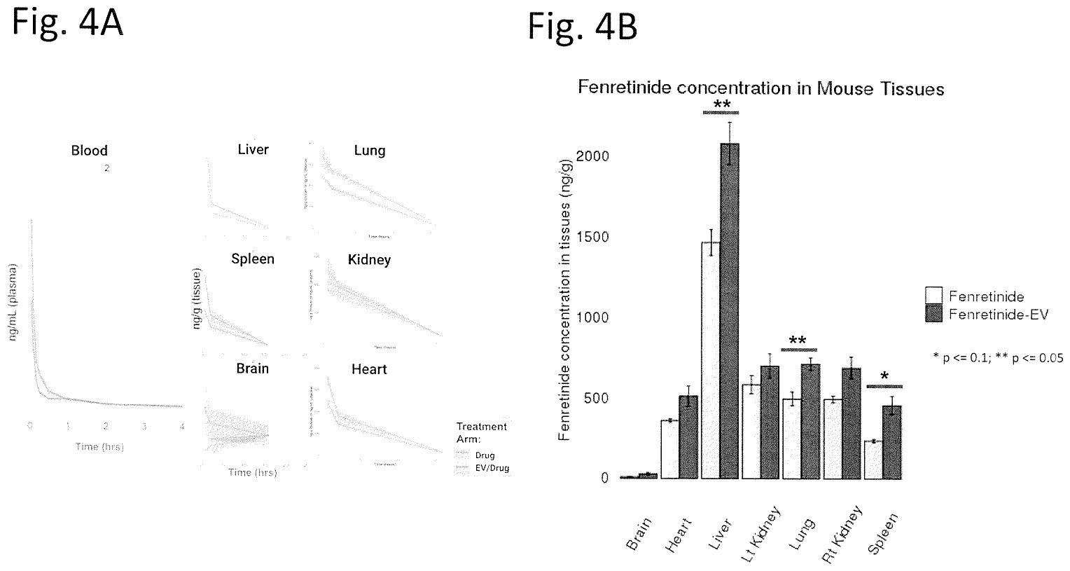

FIG. 4A shows Fenretinide concentration over time from mouse blood and tissue collections. Fenretinide-EV formulation has very fast clearance in the blood (<10% in 30 minutes) but statistically higher general tissue (e.g., liver, spleen, and lung) distributions than fenretinide-alone formulation at the early time points (e.g., 30 mins). FIG. 4B shows fenretinide concentration in blood and different tissues of CD-1® IGS mice (Charles River) at about 0.5 hr, following intravenous injection of EV-fenretinide and fenretinide formulations at a dosing level of 0.2288 mg/kg (i.e., a dose of 5.72 μg fenretinide to a/25 g CD-1® IGS mouse) . Quantification of fenretinide was by LC-MS of cell lysates prepared from blood and tissue collected from these mice.

### EXAMPLE 7: Toxicology Measurements from Tissues of Mice Injected with EV-Fenretinide Formulations

Fenretinide-EVs and fenretinide-alone formulations were produced and dosed in the animal model as in Example 6. On the terminal blood draw of each animal, 20 μL of whole blood samples were used to measure complete blood count (CBC) on the Veterinary Hematology Analyzer (Hematrue, Heska).

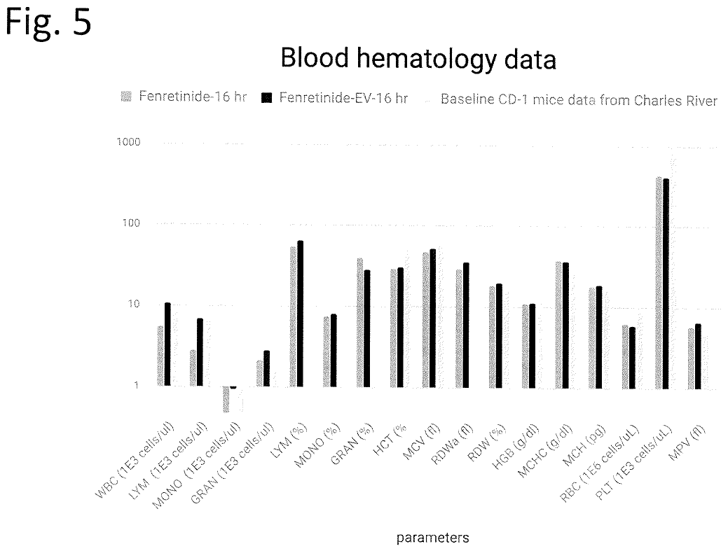

FIG. 5 shows hematology data of mice of Example 6 dosed for 16 hrs with fenretinide-alone or fenretinide-EV. Drug substance=fenretinide. X-axis: comparative scale for accepted hematology levels. Light gray bars are the Contract Research Organization provider Charles River's standards for general levels in mice. No major differences in EV-fenretinide vs fenretinide were observed. The major difference was a 5-fold decrease in PLT (platelet concentration) in both fenretinide and fenretinide-EV samples compared to general levels as reported by Charles River. Note that log scale for the y-axis.

In FIG. 5, the X-axis terms are defined as follows: WBC=white blood cell count. LYM=lymphocyte count. MONO=monocyte count. GRAN=granulocyte count. HCT=hematocrit count. MCV=mean corpuscular volume. RDW=cell blood cell distribution. HGB=Hgb protein level. MCHC=mean corpuscular hemoglobin concentration. MCH=mean corpuscular hemoglobin. RBC=red blood cell count. PLT=platelet count. MPV=mean platelet volume.

### EXAMPLE 8: Stability of Packaged Fenretinide-EV Formulations

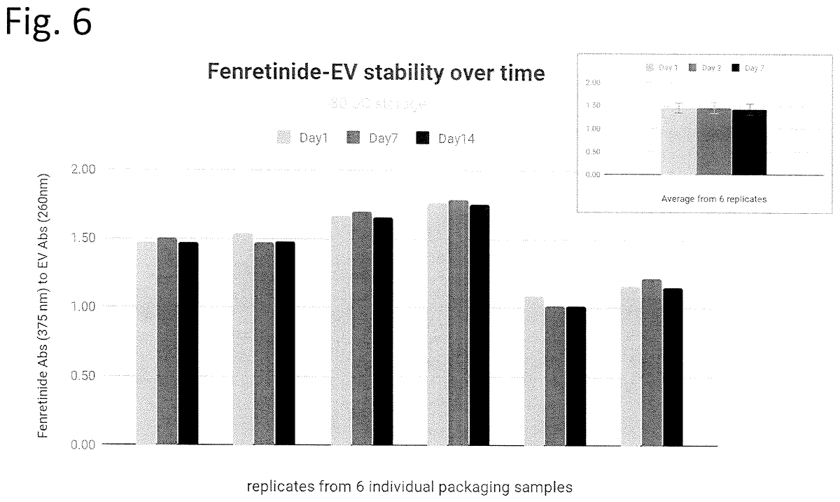

EVs were harvested, isolated and packaged with fenretinide using the protocol from Example 1. Individual EV-fenretinide vials were frozen at −80° C. and then 6 vials each were thawed on day 1, day 7 and day 14 post-freeze. The amount of fenretinide was measured via absorption spectra plate reader. FIG. 6 shows the stability of packaged Fenretinide-EV formulations over time. The fenretinide-to-EV absorption ratio (375 nm/260 nm) remained stable between days 1, 7 and 14, indicating stability of fenretinide-EV to freezing and storage at −80° C. Furthermore, the prepared and purified fenretinide-EV is stable not only to freezing but also to thawing to room temperature. The EV-fenretinide formulation using the protocol mentioned in this example produced a stable therapeutic formulation.

### EXAMPLE 9: Drug Packaging of Fenretinide in EVs Using Electroporation Technique

Suspension EVs were prepared at a concentration of 1.6E10 particles/mL. Fenretinide (Selleck Chemicals: Cat No: 55233) was dissolved in 100% DMSO as a solvent. The Fenretinide/DMSO stock solution can be 5 mM or 10 mM. MaxCyte's electroporation buffer and cartridges (OC-100×2 processing assembly) were used for electroporation and following the manufacturer recommended protocol. The Amicon® Ultra-15 10 kDa centrifugal filter (Sigma UFC901024) was used for buffer exchange of EVs into electroporation buffer. The Amicon® Ultra-0.5 10 kDa (Sigma UFC5010) was used to clean up free drug (i.e., free of EV or not in or at EV) after electroporation. Packaging of EV-fenretinide formulation comprised the following steps:

A. Buffer exchange of EVs into electroporation buffer:
1. (pre-clean Amicon® Ultra centrifugal filter) 15 mL of the electroporation buffer was added to rinse the Amicon® Ultra-15 tube. Spin at 3,000×g for 10 mins. The flow through was removed and discarded.
2. Buffer exchange was performed three times with MaxCyte vendor supplied electroporation buffer (HyClone, Cat #EPB1).
   a. (wash 1) Electroporation buffer was added to the Amicon® Ultra-15 tube. All of the EV solution was transferred to the Amicon® Ultra-15 tube and spun at 3,000×g for 15 mins or longer. The flow through was removed and discarded.
   b. (wash 2) Electroporation buffer was added to the Amicon® Ultra-15 tube, and EVs mixed well by pipetting. The Amicon® Ultra-15 tube was spun at 3,000×g for 15 mins or longer. The flow through was removed and discarded.
   c. (wash 3) Electroporation buffer was added to the Amicon® Ultra-15 tube, and EVs mixed well by pipetting. The Arnicon® Ultra-15 tube was by electroporation buffer and spun at 3,000×g for 15 mins or longer. The flow through was removed and discarded.
3. Electroporation buffer was added to the collection tube to bring the EV concentration to 1.7E11 particles/mL in the electroporation buffer, and P200 Pipetman® was used to mix and transfer the EV suspension.

B. Exosome/drug substance mixing:
1. After buffer exchange, 100 μL of the exosome solution (1.6E10 particles) was mixed with either 3 μL or 6 μL of 10 mM fenretinide/DMSO stock to give final fenretinide concentration of 300 μM or 600 μM
2. The EVs were mixed with the DMSO-dissolved fenretinide by pipetting up and down a couple of times and proceeding to electroporation steps.

C. Electroporation (EP):
1. The fenretinide/EV mixtures were transferred into the OC-100×2 (MaxCyte®) processing assembly.
2. The assembly was inserted into the electroporator, and the required EP protocol from the MaxCyte software was selected.

D. Amicon® Ultra-0.5 centrifugal filter for removing free/non-packaged drug:
4. (pre-clean Amicon® Ultra-0.5 centrifugal filter) 0.5 mL of PBS was added to rinse the

Amicon® Ultra-0.5 centrifugal filteration tube and spun at speed between 2,000 to 5,000 RPM for 10 mins. Flow through was removed and discarded.
5. Three buffer exchanges to PBS were performed.
   b. (wash 1) ˜100 μL of the positive and negative controls and electroporated fenretinide/EV mixture was transferred to each Amicon® Ultra-0.5 tube. The volume was brought up with PBS to 0.5 mL, and the sample mixed and spun at speed between 2,000 to 5,000 RPM for 10 mins. Flow through was removed and discarded.
   c. (wash 2) PBS was added to bring up the volume to 0.5 mL, and the sample mixed well by pipetting up and down and spun at speed between 2,000 to 5,000 RPM for 10 mins. Flow through was removed and discarded.
   d. (wash 3) PBS was added to bring up the volume to 0.5 mL, and the sample mixed well by pipetting up and down and spun at speed between 2,000 to 5,000 RPM for 10 mins. Flow through was removed and discarded.
6. PBS was added to the collection tube to bring the final volume back to the original sample volume to ˜100 uL if required (or spun again until the desired concentration or volume were achieved). P200 Pipetman® was used to transfer the sample.

E. Post-packaging QC metrics:
1. Drug substance quantitation: Final drug substance amount, concentration and packaging efficiency were quantified from each final drug substance packaged EV sample. Final drug substance quantitation using absorption spectra from the plate reader was measured as follows.
   a. 50 μL of the final fenretinide/EV sample was pipetted into a UV-transparent plate (Corning, cat no 3635)
   b. 2× serial dilution of the fenretinide/100% DMSO stock was performed to generate a standard curve for the drug substance (from 700 μM to 2.73 μM) and also ensuring that the standards also have 50 μL in each well.
   c. The absorption spectra (250 to 800 nm range) was measured by plate reader.
   d. The linear fit from the standard curve built from the Fenretinide/50% DMSO/50% PBS samples was used to back-calculate the concentration of fenretinide in the fenretinide/EV samples.
2. Size measurements: Samples were run on Nanoparticle Tracking Analysis (NTA) Malvern NanoSight NS300 for checking size and particle concentration (particles/mL).

### Example 10: TEM Imaging of EV-Fenretinide Samples

EV and EV-fenretinide samples were produced and prepared in a manner similar to Example 1 and stored at 4° C. for approximately two weeks. EV controls and EV-fenretinide samples are then imaged via transmission electron microscopy (TEM) to observe whether EV-fenretinide samples have different morphologies than EVs alone.

FIG. 7A and FIG. 7B show TEM images of EVs and EV-fenretinide formulations, respectively. 5 uL of EV-only preparation (FIG. 7A) and EV-fenretinide preparation (FIG. 7B) in 1×PBS were deposited on the mesh grid for 30 seconds, then stained with uranyl acetate for 1 minute and gently blotted to remove excess liquid. Images were acquired with a JEOL 1200 EX TEM instrument and showed qualitative differences between the EV-only and EV-fenretinide preparations. Specifically, the EV-only preparation (FIG. 7A) shows the typical “cup-shape” structure expected for exosomes and extracellular vesicles, whereas the EV-fenretinide preparation (FIG. 7B) shows a “walnut-like” appearance for the fenretinide-EVs, consistent with the membrane being a more membrane structure following incorporation of fenretinide into EVs. These images indicate that the EVs undergo a morphological transformation brought on by fenretinide packaging. EVs packaged with fenretinide (FIG. 7B) also appear rounder and are observed to be more structurally stable during acquisition of the TEM images. Thus, TEM distinguishes EV alone from fenretinide-EV, consistent with differences in the composition of EV and fenretinide-EV with the latter differing from the former in additionally comprising fenretinide. The EV-fenretinide formulation is indeed a distinct formulation different from stand-alone EVs, and this difference in formulation is apparent at the ultra-structural level as visualized by TEM.

### Example 11: HPLC Measurement of Non-EV Associated Fenretinide or Free Fenretinide

The amount of non-EV associated fenretinide, or free fenretinide, in final EV-fenretinide samples, was measured via HPLC after collecting different fractions of the EV-fenretinide samples passed through a size-exclusion chromatography column (SEC). Each fraction collection volume was 0.2 mL. A solvent of 0.1% trifluoroacetic acid (TFA) in ethanol was used in order to solubilize fenretinide and fenretinide aggregates and disrupt and solubilize fenretinide-EV on HPLC. The SEC fraction samples were diluted 3× in 0.1% TFA in ethanol diluent, and chromatography was performed on an HPLC system. Because the major lipid content of EVs is cholesterol, the amount of cholesterol in the EV-Fenretinide sample was used as a proxy for the EVs. Quantitation of the amount of cholesterol and fenretinide in the various SEC column fractions via HPLC and plotting the quantified values for cholesterol and fenretinide based on SEC column fraction number result in the plot shown in FIG. 8. A strong correlation is seen between fenretinide and cholesterol content for the fractions that passed through SEC filtration. This is consistent with tight packaging of fenretinide onto the EV surface or inside the lipid bilayer. Furthermore, this analysis is consistent with an earlier analysis demonstrating little, if any, free fenretinide—fenretinide free of extracellular vesicle in the final purified extracellular vesicle samples. Fenretinide in the fenretinide-EV formulation appears stably associated with EVs.

### Example 12: EVs from Mesenchymal Stem Cells (MSCs)

EVs can be obtained from mesenchymal stem cells (MSCs), also called mesenchymal stromal cells, cultured in suspension bioreactors and then packaged with fenretinide, as in Example 1, 2, 3, 9 or 13. They can be stored at −80° C. for three weeks until a pediatric neuroblastoma subject is ready to be treated.

### Example 13: Large Scale Packaging Protocol

This protocol allowed packaging of 680.8 μg of drug substance inside EV (6.83E11 particles) with a 51.2% encapsulation efficiency. This protocol used Amicon® Ultra-15 diafiltration concentrators for larger batch production to produce the batch sizes necessary for in vivo animal studies. In another embodiment, similar protocols could be developed for small batch sizes using smaller Amicon® Ultra diafiltration concentrators, such as Amicon® Ultra-4 and Amicon® Ultra-0.5 centrifugal filters.

Packaged EVs were then split for two separate studies and further diluted from fenretinide concentration of about 345.9 μM (calculated from absorbance spectrum obtained from a plate reader) in the fenretinide-EV with non-packaged EVs in about 1×PBS (1.5E11 particles/ml) to the final fenretinide-EV formulation with fenretinide concentration at about 134.5 μM and particle concentration at about 1.28E11 particles/ml for the rat laser CNV study design. This final Fen-EV packaged sample was then dosed on rats.

Packaged EVs (i.e., fenretinide-EV) at a fenretinide concentration of 345.9 μM were diluted to a final formulation with 134.5 μM fenretinide concentration and 1.28E11 particles/mL EV particle concentration. About 5 μL of the final fenretinide-EV formulation were administered by intravitreal injection to each eye of a rat subject in a rat model of laser-induced choroidal neovascularization (CNV).

EVs were prepared at a concentration of 1.5E+11 particles/mL in PBS. Fenretinide (Selleck Chemicals: Cat No: 55233) was dissolved in 100% DMSO. The fenretinide/DMSO stock solution can be 5 mM or 10 mM.

The following protocol was used for packaging the EV-Fenretinide formulation.

A. Exosome/drug substance incubation:
1. EV samples were diluted with PBS to have a final EV concentration around 1.5E11 particles per mL.
2. For every 10,000 μL of EV sample in PBS, 344.79 μL of 10 mM fenretinide in DMSO was added to achieve a drug substance mass (in μg) to 1E9 EV count ratio of 0.9 in the appropriate tube format. (Final DMSO concentration for this sample was ˜3.3% (vol/vol)).
3. The EVs were mixed with the drug solution by pipetting up and down a couple of times, with no incubation, then processed immediately according to the clean-up steps below.

B. Corning® Costar® SpinX® (0.22 μm cellulose acetate membrane filter) centrifuge tube filter for large drug aggregates removal
1. About 10,000 μL of fenretinide-EV samples were split into four 2.5 mL Corning® Costar® SpinX® 0.22 μm cellulose acetate filters (˜2,500 μL in each) and filtered by centrifugation at 1,000×g for 5 minutes.
2. Samples were pooled to obtain ˜10 mL volume and small aliquots (e.g., 5 μL) were collected pre- and post-filtration for in-process analysis.

C. Amicon Ultra-15 centrifugal filter for removing free/non-packaged drug substance:
1. 15 mL of 1×PBS buffer were added to rinse the 10 kD Arnicon® Ultra-15 concentrator tube (Sigma/Millipore) and centrifuged at 3,220×g for 15 mins. The flow-through was discarded.
2. (Wash 1) 13 mL of 1×PBS was added to each concentrator tube. 2 mL of filtered fenretinide/EV sample were added to the concentrator tube and centrifuged at 3,220×g until the sample was concentrated to ˜1.5 mL. The flow through was discarded.
3. (Wash 2) 15 mL of PBS was added to each concentrator tube and mixed well by pipetting. The tube was centrifuged at 3,220×g until sample and was concentrated to ˜1.5 mL. The flow through was discarded.
4. (Wash 3) 15 mL of PBS was added to each concentrator tube and mixed well by pipetting. The tube was centrifuged at 3,220×g until sample and was concentrated to ˜1.5 mL. The flow through was discarded.
5. P1000 Pipetman® was used to rinse the concentrator membrane of each tube followed by a P200 Pipetman® to ensure all material was transferred. The concentrated sample was transferred to 2×2.5 ml 0.22 μm cellulose acetate Spin-X® centrifugal tube filters to filter by centrifuging at 1,000×g for 5 minutes, resulting in a final volume of 5,100 μL.
6. Absorption spectra measurement (using Synergy, Biotek) performed for fenretinide-EV sample and standard curve for fenretinide in DMSO/PBS—200 μL, 1:5 Dilution Fen-EV:PBS (fenretinide in DMSO/PBS standard curve) to determine fenretinide concentration present in fenretinide-EV sample.
7. NTA measurement was performed (5 μL fenretinide-EV added into 1 mL of filtered PBS).
8. Based on the concentration determined in Step 6, diluted the sample to a target concentration of 150 μM fenretinide using the starting EV sample (1.5E11 particles/ml) in 1×PBS.
9. The diluted sample was transferred to 2×2.5 mL 0.22 um Spin-X filters to filter by centrifuging at 1000 g for 5 minutes to obtain a final volume of 5800 uL.
10. Analyzed absorption spectra measurement (using Synergy H1 plate reader)—200 uL, 1:5 Dilution Fen-EV:PBS (fenretinide in DMSO/PBS standard curve generated the previous day).
11. Analyzed NTA measurement (5 uL), used Camera Level 14.

### Example 14: ARPE-19 cell viability with EV-fenretinide dosing

EV-fenretinide was packaged and purified in a manner similar to the protocol described in Example 1. The extracellular vesicles used for packaging fenretinide was isolated from HEK293-F cells. A human retinal pigment epithelium (RPE) cell line, ARPE-19 (ATCC® CRL-2302™), isolated from normal eyes of an adult male, is treated with fenretinide alone or fenretinide-EV to examine the effect on viability of retinal pigment epithelium cells. ARPE-19 cells plated the night before were treated with fenretinide (resuspended in 100% DMSO) or fenretinide-EV (in PBS) at matched concentration of drug substance (active pharmaceutical ingredient (API)) dosed at either low (0.94 μM) or high (15 μM) fenretinide concentration. After 48 hrs at 37° C. in a humidified 5% CO₂ incubator, cell viability is measured using CellTiter-Huor™ Cell Viability Assay (Promega) following the manufacturer's instructions to detect viable cells based on cell membrane integrity using a fluorogenic, cell-permeant, peptide substrate (Gly-Phe-AFC) which is cleaved to a fluorescent signal following entry into intact cells. The plate was read on plate reader at 380-400 nm excitation and —505 nm emission for live-cell fluorescence readout.

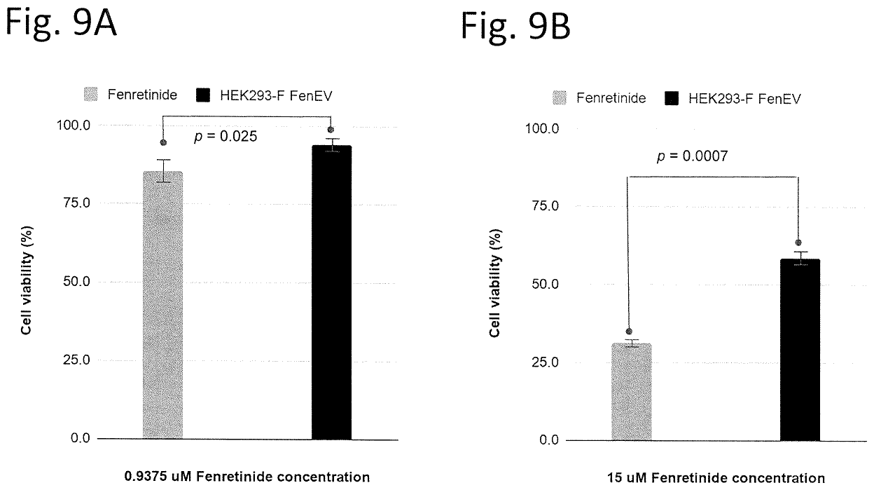

FIG. 9 shows ARPE-19 treated with fenretinide-EV has less growth inhibition (e.g., more viable cells) at both low and high concentration than fenretinide alone in eye cells. At a low concentration corresponding to 0.94 μM fenretinide equivalent (FIG. 9A), treatment with fenretinide alone resulted in 85.5% cell viability or growth of control, untreated ARPE-19 cells, while treatment with fenretinide-EV resulted in 94.3% cell viability or growth—a percentage point difference of 8.8%. At a higher concentration corresponding to 15 μM fenretinide equivalent (FIG. 9B), treatment with fenretinide alone resulted in 31% cell viability or growth of untreated ARPE-19 cells, while treatment with fenretinide-EV resulted in 58.6% cell viability or growth—a percentage point difference of 27.6%. The greater cell viability achieved with fenretinide-packaged in extracellular vesicles (fenretinide-EVs) provides an advantage over fenretinide alone when fenretinide is used as a drug substance or API to treat eye cells.

### Example 15: Retention and Biodistribution of Fenretinide in Eye After Intravitreal Injection of Fenretinide or Fenretinide-EV Formulations

Eye biodistribution of fenretinide formulated as 90.9 uM free fenretinide (excipient: 5% Ethanol, 12.5% Solutol-HS 15, 12.5% PEG 300, and 70% PBS) or 94.8 uM fenretinide-EV (in PBS) is administered to each eye of brown Norway rat (6 rats/treatment condition) by intravitreal injection (5 μL per eye). 24-hrs after administration, eyes were enucleated. Individual tissues were collected corresponding to cornea, conjunctiva, lens, vitreous humor, retina, RPE/choroid and sclera and analyzed for fenretinide content. In addition, total fenretinide content in each eye was also determined.

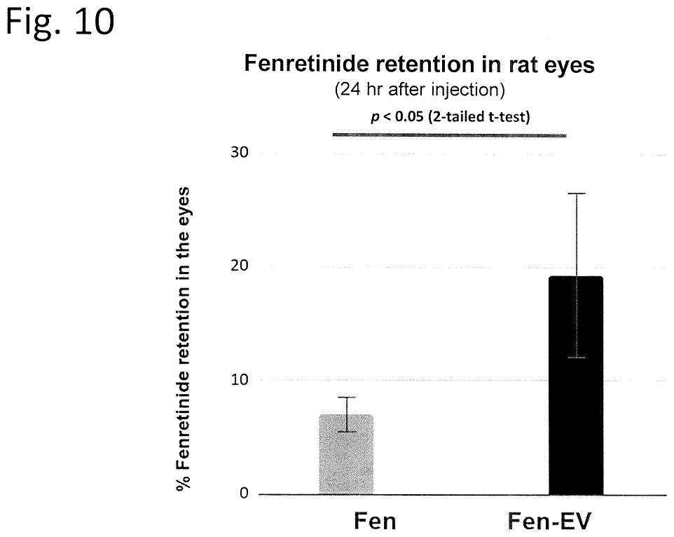

FIG. 10 shows averages of the total fenretinide content remaining in eyes of 6 brown Norway rats 24-hrs after intravitreal injection of fenretinide alone or fenretinide-EV formulation. Note the presence of a greater percentage of fenretinide present for eyes receiving fenretinide-EV than fenretinide alone (19% vs. 7%), consistent with greater eye retention of fenretinide when administered as fenretinide-EV than fenretinide alone.

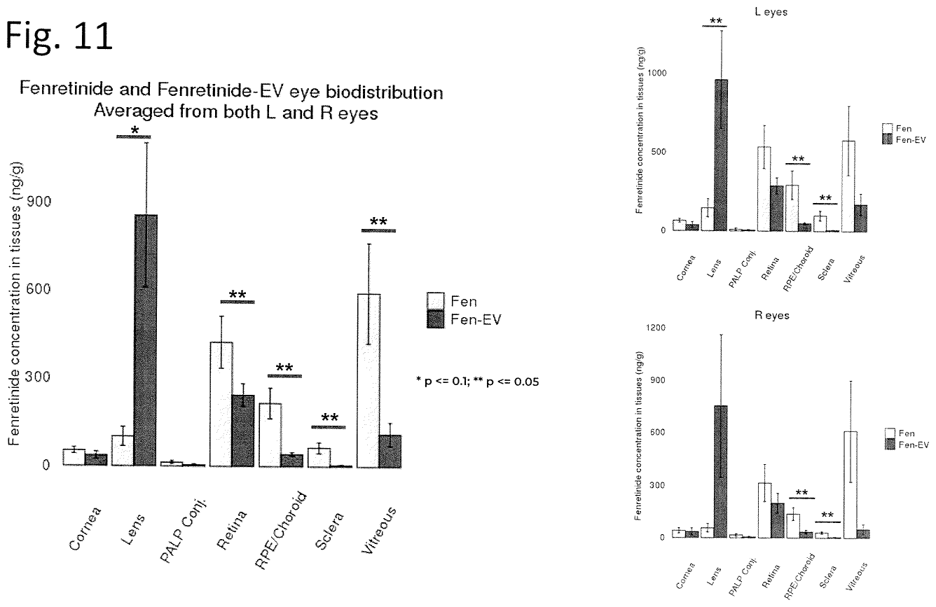

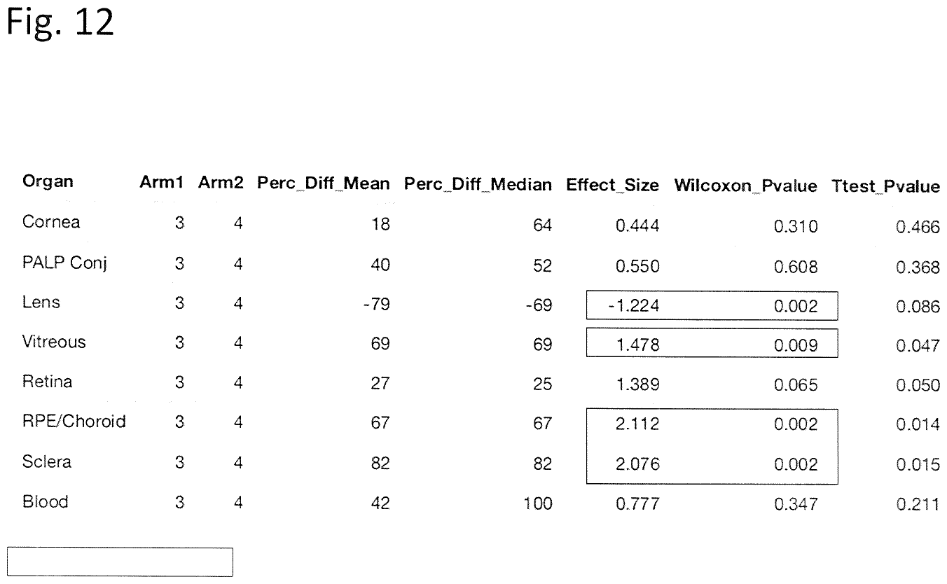

FIG. 11 shows concentration of fenretinide (ng fenretinide per gram rat tissue weight) averaged for different eye tissues isolated from left eye (L eye), right eye (R eye) or both (L+R eye) following 24-hrs after intravitreal injection of fenretinide alone or fenretinide-EV formulation in brown Norway rats (6 rats per formulation). While fenretinide can accumulate at all tissues examined (such as cornea, lens, palpebral conjunctiva, retina, retinal pigment epithelium (RPE)/choroid, sclera and vitreous humor), the relative distribution of fenretinide at the different tissues depends on fenretinide formulation used for the intravitreal injection. Most notable is the concentration of fenretinide at lens of the eyes when fenretinide is packaged in extracellular vesicles. Statistical analysis of differences in eye sub-tissue distribution between fenretinide alone formulation and fenretinide-EV formulation (based on average of two eye values from each animal) showed a statistically significant increase in fenretinide concentration at the lens and a decrease at the RPE/choroid eye tissue, sclera and vitreous humor (FIG. 12). This alteration in the biodistribution of fenretinide upon incorporation into extracellular vesicles provides a method for altering trafficking or routing fenretinide administered to an eye and also a potential for the lens to serve as a reservoir for fenretinide. Extracellular vesicles may be used to preferentially target certain eye tissues, avoid certain eye tissues or modulate drug substance retention, release and distribution in the eye.

### Example 16: Evaluation of the Effect of Fenretinide-EV Formulation on Lesion Size and Vascular Leakage Following a Single Bilateral Intravitreal Injection in a Rat Model of Laser-Induced Choroidal Neovascularization (CNV)

A 25-day study was conducted in a rat model of laser CNV (Shah, R S et al. (2015) J Vis Exp 106:e53502; Wigg, J Pet al. (2015) PLoS One 10:e0128418) to determine the potential beneficial effects of fenretinide-EV test article. Female brown Norway rats (6-8 weeks of age; 130-150 g) from Charles River Laboratories were divided into 2 separate treatment groups. On Day 1, laser-induced lesions in the Bruch's membrane were created using a 520 nm thermal laser to generate a total of three lesions per eye to all study animals. On Day 3, all study animals received a bilateral intravitreal administration of vehicle (PBS; Arm 1) or EV test article 1 (Arm 2). On Day 22, in vivo fluorescein angiography was performed. Lesion size area was determined to calculate neovascularization, and the integrated density of fluorescein intensity was determined to calculate the extent of vascular leakage within the neovascular area.

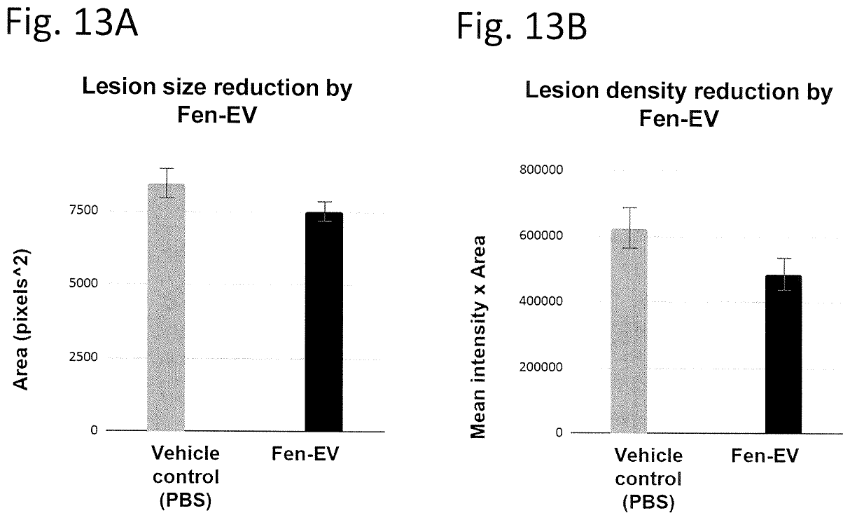

FIG. 13A shows a bar graph of lesion measurements 3-weeks post laser treatment, where treatment with fenretinide-EV appears to reduce lesion size over mock treated control (PBS). FIG. 13B shows a bar graph of integrated density of lesions 3-weeks post laser treatment, where fenretinide-EV has a more dramatic effect on reducing magnitude of the integrated density. Taken together, fenretinide-EV treatment may be effective in the treatment of diseases or conditions associated with neovascularization and vascular leakage, as is found in wet age-related macular degeneration. The data support further development of fenretinide-EV as a therapeutic agent.

### Example 17: Packaging of c-Kit Inhibitor Drugs with EVs

Both hydrophobic and hydrophilic versions of imatinib and dasatinib as c-kit inhibitor drugs were used to incorporate into extracellular vesicles. Hydrophobic versions are: imatinib and dasatinib. Hydrophilic versions are: imatinib mesylate and dasatinib hydrochloride.

TABLE 2
Types of c-kit inhibitor drugs tested for packaging with EVs accompanied
by the packaging method used for each of the drug.

| | Type of Drugs | | | |
|---|---|---|---|---|
| | Hydrophobic c-kit | | Hydrophilic c-kit | |
| | inhibitor drugs | | inhibitor drugs | |
| | Drug names | | | |
| | | | Dasatinib | Imatinib |
| | Dasatinib | Imatinib | Hydrochloride | Mesylate |
| Packaging | Incubation | Incubation | Indirect Bath | Indirect Bath |
| Protocols | | | Sonication | Sonication |
| tested | Direct Probe | Direct Probe | Active | Active |
| | sonication | sonication | Loading | Loading |
| | — | — | Electro- | Electro- |
| | | | poration | poration |

### Hydrophobic c-Kit Inhibitor Drug Packaging Protocols

### 1. Incubation Protocol (Room Temperature and 40 Degree Celsius)

For packaging using the incubation protocol, suspension EVs were prepared at a concentration of ∫2.3E11 particles/mL. Dasatinib (Selleck Chemicals Cat No.: S1021; Dasatinib |≥99% (HPLC)|Selleck|Src inhibitor) and Imatinib (Selleck Chemicals Cat No.: S2475 Imatinib (STI571) |≥99% (HPLC)|Selleck|PDGFR inhibitor) were dissolved in DMSO to make a stock solution of 10 mM. The following incubation protocol was used for packaging the EV-Dasatinib or EV-Imatinib formulation.

Exosome/drug mixing:
1. EV solution diluted in PBS, corresponding to 5.5E10 particles/sample were mixed with either Dasatinib (D) or Imatinib (I) to give final drug concentrations of either 600 μM or 300 μM.
2. The final volume before incubation was adjusted to 250 μL if required, using DMSO. The negative control (no drug) EVs, received ‘DMSO only’ to bring up the volume to 250 μL. The final DMSO content was ˜5.7%.
3. The EVs were mixed with the drug solution by pipetting up and down a couple of times and then proceeded to the incubation step.

Incubation:
1. Samples were incubated either at room temperature or 40 degrees Celsius (using a heat block) for 2 hours.
2. Post-incubation the samples were moved to the incubator at 37° C. for 15 mins, followed by incubation on ice for 15 mins to allow the EVs to recover.
3. After the EV recovery step, the free/unpackaged drug was removed from the sample following the drug clean up protocol described in the section, “Protocol for free drug clean up.”

(a) 2. Direct Probe Sonication Protocol

For packaging using the probe sonication protocol, suspension EVs were prepared at a concentration of ˜2.0E11 particles/mL. Dasatinib (Selleck Chemicals Cat No.: S1021; Dasatinib |≥99%(HPLC)|Selleck|Src inhibitor) and Imatinib (Selleck Chemicals Cat No.: S2475 Imatinib (STI571) |≥99% (HPLC)|Selleck|PDGFR inhibitor) were dissolved in DMSO to make a stock solution of 10 mM. The following probe sonication protocol was used for packaging the EV-Dasatinib or EV-Imatinib formulation.

Exosome/drug mixing:
1. EV solutions diluted in PBS, corresponding to 6E10 particles/sample were mixed with either Dasatinib (D) or Imatinib (I) to give final drug concentrations of either 600 μM or 300 μM.
2. The final volume before sonication was adjusted to 318 μL if required, using DMSO. The negative control (no drug) EVs, received ‘DMSO only’ to bring up the volume to 318 μL. The final DMSO content was ˜5.7%.
3. The EVs were mixed with the drug solution by pipetting up and down a couple of times and then proceeded to sonication.

Probe Sonication:
1. Q500 Sonicator, QSonica, LLC installed with a 1/16″ microtip was used for sonication.
2. The samples were sonicated using the following condition: 20% amplitude, 30 second pulses (6 cycles) with 59 seconds rest between each sonication cycle.
3. The samples were placed on ice during sonication to prevent the EVs from being denatured from the heat dissipated.
4. Once the samples were sonicated, they were moved to the incubator at 37° C. to allow the EVs to recover for 1 hour.
5. After the EV recovery step, the free/unpackaged drug was removed from the sample following the drug clean up protocol described in the section, “Protocol for free drug clean up.”

### Hydrophilic c-Kit Inhibitor Drug Packaging Protocols

### 1. Indirect Bath Sonication Protocol

For packaging using the indirect bath sonication protocol, suspension EVs were prepared at a concentration of ˜2.3E11 particles/mL. Hydrophilic versions of the c-kit drugs, Dasatinib Hydrochloride (Selleck Chemicals Cat No.: S5254; Dasatinib hydrochloride |≥99% (HPLC)|Selleck|Bcr-Ab1 inhibitor) and Imatinib Mesylate Selleck Chemicals Cat No.: S1026; Imatinib Mesylate (STI571) |≥99% (HPLC)|Selleck |Bcr-Ab1 inhibitor) were dissolved deionized water to make a stock solution of 10 mM.

The following indirect bath sonication protocol was used for packaging the EV-Dasatinib Hydrochloride or EV-Imatinib Mesylate formulation.

Exosome/drug mixing:
1. EV solutions diluted in PBS, corresponding to 5.5E10 particles/sample were mixed with either Dasatinib hydrochloride (DH) or Imatinib Mesylate (IM) to give final drug concentration of either 600 μM or 300 μM.
2. The final volume before incubation was adjusted to 250 μL if required, using deionized water. The negative control (no drug) EVs, received ‘Deionized water only’ to bring up the volume to 250 μL.
3. The EVs were mixed with the drug solution by pipetting up and down a couple of times and then proceeded to the sonication step.

Bath Sonication:
1. Q500 Sonicator, QSonica, LLC with a cup horn attachment was used to perform indirect bath sonication. The cup horn was filled with cold water during operation to dissipate the heat that forms during sonication, which prevents the sample from denaturing
2. The samples were sonicated using the following condition: either 50% or 80% amplitude applied in 30 second pulses (6 cycles), with 59 seconds rest between each cycle. This condition generated a constant pulse of 125 watts for 50% amplitude and 223 watts for 80% amplitude.
3. Once the samples were sonicated, they were moved to the incubator at 37° C. to allow the EVs to recover for 1 hour.
4. After the EV recovery step, the free/unpackaged drug was removed from the sample following the drug clean up protocol described in the section, “Protocol for free drug clean up.”

### 2. Active Loading Protocol

For packaging using the active loading protocol, suspension EVs were prepared at a concentration of ˜2.8E11 particles/mL. Hydrophilic versions of the c-kit drugs, Dasatinib Hydrochloride (Selleck Chemicals Cat No.: S5254; Dasatinib hydrochloride|≥99% (HPLC)|Selleck|Bcr-Ab1 inhibitor) and Imatinib Mesylate (Selleck Chemicals Cat No.: S1026; Imatinib Mesylate (STI571) |≥99% (HPLC)|Selleck|Bcr-Ab1 inhibitor) were dissolved deionized water to make a stock solution of 10 mM.

The following active loading protocol was used for packaging the EV-Dasatinib Hydrochloride or EV-Imatinib Mesylate formulation.

Buffer Exchange of EVs in to Ammonium Sulphate buffer using Amicon® Ultra-15 concentrator tube (30 kDa):
1. EV solution corresponding to 11.2E11 total particles was buffer exchanged against 150 mM Ammonium Sulphate buffer pH 5.5.
2. 3× buffer exchange of EVs was performed following steps below:
   a. (wash 1) EV solution was added to a pre-washed Amicon® Ultra-15 concentrator tube. The volume was brought up to ˜15 mL with the Ammonium Sulphate buffer. After mixing, the Amicon® tubes were spun at a speed of 3,220×g for anywhere between 10-25 mins until the solution in the tube came down to ˜3 mL. The flow through was discarded.
   b. (wash 2) The contents of the tube were mixed by pipetting up and down and ammonium sulphate buffer was added to bring up the volume to 15 mL. After mixing, the tubes were spun again at a speed of 3,220×g for anywhere between 10-25 mins until the solution in the tube was ˜3 mL. The flow through was discarded.
   c. (wash 3) The Process in step b was repeated once more, for the final wash.

Ammonium sulphate entrapment in the core:
1. The EV samples were then transferred to a 40° C. heat block for 60 mins to encapsulate the ammonium sulphate into the core of EVs.
2. After an hour, the EVs were first moved to the incubator at 37° C. for ˜20 mins and then on ice for ˜20 mins for membrane recovery, before proceeding to buffer exchange into 0.9% NaCl solution.

Buffer Exchange of EVs in to 0.9% NaCl: (This step creates the ion gradient to drive drug loading)
1. The buffer exchange steps were repeated exactly following the same protocol used for ammonium sulphate buffer exchange using Amicon® Ultra-15 concentrator tube (30 kDa), but at this step the equilibration buffer used was 0.9% NaCl.
2. At this point, the EVs are ready for drug packaging.

Exosome/drug mixing:
1. EV solutions in 0.9% NaCl, were then mixed with either Dasatinib hydrochloride (DH) or Imatinib Mesylate (IM) to give final drug concentrations of either 600 μM or 300 μM.
2. The final volume before incubation was adjusted to 265 μL if required, using deionized water. The negative control (no drug) EVs, received ‘Deionized water only’ to bring up the volume to 265 μL.
3. The EVs were mixed with the drug solution by pipetting up and down a couple of times.
4. Samples were incubated at 40° C. on a heat block for 2 hours.
5. After 2 hours at 40° C., the EVs were moved to the incubator at 37° C. for 20 mins followed by incubation on ice for 20 mins to allow the EV membranes to recover.
6. After the EV recovery step, the free/unpackaged drug was removed from the sample following the drug clean up protocol described in the section, “Protocol for free drug clean up.”

### (b) 2. Electroporation Protocol

For packaging using the indirect bath sonication protocol, suspension EVs were prepared at a concentration of ˜2.5E11 particles/mL. Hydrophilic versions of the c-kit drugs, Dasatinib Hydrochloride (Selleck Chemicals Cat No.: S5254; Dasatinib hydrochloride |≥99% (HPLC)|Selleck|Bcr-Ab1 inhibitor) and Imatinib Mesylate (Selleck Chemicals Cat No.: S1026; Imatinib Mesylate (STI571) |≥99%( HPLC)|Selleck |Bcr-Ab1 inhibitor) were dissolved deionized water to make a stock solution of 10 mM. The following electroporation protocol was used for packaging the EV-Dasatinib Hydrochloride or EV-Imatinib Mesylate formulation.

Buffer Exchange of EVs into electroporation buffer:
1. EV solution corresponding to 2.5E+11 particles/mL was buffer exchanged against the electroporation buffer supplied by MaxCyte using an Amicon® Ultra-15 concentrator.
2. 3× buffer exchange was performed following steps below—
   a. (wash 1) EV solution was added to a pre-washed Amicon® Ultra-15 tube. The volume was brought up to ˜15 mL with the electroporation buffer. After mixing, the Amicon® Ultra-15 tube was spun at a speed of 3,220×g for anywhere between 10-25 mins. The flow through was discarded.
   b. (wash 2) The contents of the tube were mixed by pipetting up and down and the electroporation buffer was added to bring up the volume to 15 mL. After mixing, the tubes were spun again at a speed of 3,220×g for anywhere between 10-25 mins. The flow through was discarded.
   c. (wash 3) Process in step b was repeated once more for the final wash.

Exosome/drug mixing:
1. After buffer exchange, EV solution in electroporation buffer corresponding to 5.5E10 particles/sample were mixed with either Dasatinib Hydrochloride or Imatinib Mesylate to give final drug concentrations of 300 μM, 600 μM, 900 μM, 3 mM, 6 mM and 9 mM.
2. The final volume before electroporation was adjusted to 260 μL if required, using the EP buffer.
3. EVs were mixed with the drug solution by pipetting up and down a couple of times and then proceeded to electroporation steps.

Electroporation (EP):
1. The drug/EV mixtures were transferred into the OC-4×100 processing assembly provided by Maxcyte.
2. The assembly was inserted into the machine and EP protocol 56 was chosen in the MaxCyte software. The electroporation takes a couple of seconds.
3. After EP, the EVs were then allowed to recover at RT for 20 mins before proceeding to free drug clean-up steps as per “Protocol for free drug clean up.”

### Protocol for Free Drug Clean Up

After carrying out any of the packaging protocols described above, the free/unpackaged drug was removed from the EV samples, in case of both, the hydrophilic and hydrophobic versions of Dasatinib and Imatinib, using the protocol below—

Step 1: Corning® Costar® SpinX® cellulose acetate filter (0.22 μm) for removal of large drug aggregates:
1. The packaged EV samples were first filtered through 0.22 μm Corning® Costar® SpinX® cellulose acetate filters by centrifugation at 5,000 RPM for 10 minutes to remove any large drug or drug-EV aggregates.

Step 2: Amicon Ultra-0.5 centrifugal filter (10 kDa) for removing free/non-packaged drug:
1. Next, a 3× buffer exchange of packaged EVs into PBS was performed.
   a. (wash 1) ˜250 to 300 μL of the sample was added to an Arnicon® Ultra-0.5 tube. The volume was brought up with 1×PBS to ˜0.5 mL. After mixing, the samples were spun at speed of ˜5,000 RPM for anywhere between 10-25 mins. The flow through was discarded.
   b. (wash 2) PBS was added to bring up the volume to 0.45 mL. Samples were mixed well by pipetting. Samples were spun at speed of ˜5,000 RPM for anywhere between 10-25 mins. The flow through was discarded.
   c. (wash 3) PBS was added to bring up the volume to 0.45 mL. Samples were mixed well by pipetting. Samples were spun at speed of ˜5,000 RPM for anywhere between 10-25 mins. The flow through was discarded.
2. After the washes, PBS was added to the collection tube to bring the final volume to the desired sample volume if required (or spun again until the desired volume was achieved).

Step 3: Final Sterilization using Corning Costar® SpinX® cellulose acetate filter (0.22 μm):
1. Finally, the samples were filtered through a 0.22 μm Costar SpinX cellulose acetate filter by centrifugation at 5,000 RPM for 15 mins. Filtered volumes were around ˜250 μL.
2. Samples were stored in 4° C. overnight until further analysis for drug content determination by absorption using a plate reader assay and NTA for particle concentration.

### Post-Packaging QC Metrics

1. Drug quantitation: Drug Concentration and packaging efficiency were quantified for the final drug packaged EV sample. Final drug quantitation was performed using absorption spectra from the plate reader using the following steps:
   a. Quantification of Dasatinib or Dasatinib hydrochloride in drug/EV samples-
      i. 150 μL of the final Dasatinib or Dasatinib Hydrochloride packaged EVs were mixed with 200 μL of MeOH to extract the packaged drug for detection.
      ii. This mixture was incubated at −20° C. for 1 hour to aid in the extraction of the drug and then spun at 15,000×g for 15-20 mins.
      iii. The supernatant solution was moved directly to a low UV absorbance 96-well plate without further dilution at 150 μL/well in duplicates.
      iv. The concentration of Dasatinib or Dasatinib Hydrochloride in the samples was estimated from a standard curve of the respective drug in methanol, in the concentration range of 0.00580 mM-0.00002 mM with 2× serial dilution.
      v. The spectra was measured from about 230 nm to 800 nm with the peak appearing at about 320 nm for both Dasatinib and Dasatinib Hydrochloride.
   b. Quantification of Imatinib or Imatinib Mesylate in drug/EV samples—
      i. 150 μL of the final Imatinib or Imatinib Mesylate packaged EVs were mixed with 200 μL of MeOH to extract the packaged drug for detection.
      ii. This mixture was incubated at −20° C. for 1 hour to aid in the extraction of the drug and then spun at 15,000×g for 15-20 mins.
      iii. The supernatant solution was moved directly to a low UV absorbance 96-well plate without further dilution at 150 μL/well in duplicates.
      iv. The concentration of Imatinib or Imatinib Mesylate in the samples was estimated from a standard curve of the respective drug in methanol in the concentration range of about 0.188 mM-0.0007 mM with 2× serial dilution.
         v. The spectra was measured from about 230 nm to 800 nm range with the peak appearing at about 270 nm for both Imatinib and Imatinib Mesylate.
2. Size measurements: Samples were run on Nanoparticle Tracking Analysis (NTA) NanoSight NS300 (Malvern) for checking size and particle concentration (particles/mL)

### Drug Packaging Results

TABLE 3
Drug concentration, Packaging Efficiency and Particle concentration for final packaged
samples with Hydrophobic c-kit inhibitor drugs.

| | | | Final Drug | | | Average | |
|---|---|---|---|---|---|---|---|
| | Drug name | | Packaging/ | | Final Total | median | |
| | and starting | Final Drug | Encapsulation | Final Particle | particles | particle size | Drug |
| Packaging | drug | concentration | Efficiency | concentration | (Particle | (nm) ± med se | molecules |
| method used | concentration | (μM) | (%) | (Particles/mL) | number) | % | per EV |
| Incubation at | Dasatinib- | 5.22 | 1.74 | 7.38E+10 | 1.85E+10 | 137.1 ± 0.15 | 4.26E+04 |
| room | 300 μM | | | | | | |
| temperature | Dasatinib- | 8.03 | 1.34 | 2.44E+10 | 6.10E+09 | 128.9 ± 0.62 | 1.98E+05 |
| | 600 μM | | | | | | |
| | Imatinib- | 19.19 | 6.40 | 8.70E+10 | 2.18E+10 | 139.2 ± 0.50 | 1.33E+05 |
| | 300 μM | | | | | | |
| | Imatinib- | 42.13 | 7.02 | 6.58E+10 | 1.65E+10 | 134.3 ± 0.60 | 3.86E+05 |
| | 600 μM | | | | | | |
| Incubation at | Dasatinib- | 4.47 | 1.49 | 8.20E+10 | 2.05E+10 | 137.6 ± 0.22 | 3.29E+04 |
| 40 degree | 300 μM | | | | | | |
| celcius | Dasatinib- | 3.89 | 0.65 | 7.74E+10 | 1.94E+10 | 135.7 ± 1.62 | 3.03E+04 |
| | 600 μM | | | | | | |
| | Imatinib- | 17.37 | 5.79 | 6.60E+10 | 1.65E+10 | 138.2 ± 0.65 | 1.59E+05 |
| | 300 μM | | | | | | |
| | Imatinib- | 37.63 | 6.27 | 2.09E+10 | 5.23E+09 | 127.3 ± 1.49 | 1.08E+06 |
| | 600 μM | | | | | | |
| Direct Probe | Dasatinib- | 0.61 | 0.20 | 1.14E+11 | 3.42E+10 | 133.40 ± 0.82 | 3.23E+03 |
| sonication | 300 M | | | | | | |
| (20% | Dasatinib- | 3.61 | 0.60 | 9.01E+10 | 2.70E+10 | 128.90 ± 0.47 | 2.41E+04 |
| Amplitude) | 600 μM | | | | | | |
| | Imatmib- | 18.67 | 6.22 | 9.70E+10 | 2.91E+10 | 130.20 ± 0.77 | 1.16E+05 |
| | 300 μM | | | | | | |
| | Imatinib- | 25.99 | 4.33 | 1.09E+11 | 3.27E+10 | 136.90 ± 1.02 | 1.44E+05 |
| | 600 μM | | | | | | |

TABLE 4
Drug concentration, Packaging Efficiency and Particle concentration for final packaged samples with
Hydrophilic c-kit inhibitor drugs; DH-Dasatinib Hydrochloride and IM-Imatinib Mesylate.

| | | | Final Drug | | | Average | |
|---|---|---|---|---|---|---|---|
| | Drug name | | Packaging/ | Final | Final Total | median | |
| | and starting | Final Drug | Encapsulation | Particle | particles | particle size | Drug |
| Packaging | drug | concentration | Efficiency | concentration | (Particle | (nm) ± med se | molecules |
| method used | concentration | (μM) | (%) | (Particles/mL) | number) | % | per EV |
| Indirect Bath | DH-300 μM | 3.69 | 1.23 | 4.96E+10 | 1.24E+10 | 108.50 ± 0.55 | 4.48E+04 |
| Sonication | DH-600 μM | 2.46 | 0.41 | 1.20E+10 | 3.00E+09 | 123.70 ± 2.18 | 1.23E+05 |
| (50% | IM-300 μM | 20.10 | 6.70 | 7.30E+10 | 1.83E+10 | 114.00 ± 1.14 | 1.66E+05 |
| Amplitude | IM-600 μM | 19.53 | 3.26 | 6.70E+10 | 1.68E+10 | 110.10 ± 1.91 | 1.76E+05 |
| Indirect Bath | DH-300 μM | 8.04 | 2.68 | 6.14E+10 | 1.54E+10 | 111.30 ± 0.99 | 7.89E+04 |
| Sonication | DH-600 μM | 2.95 | 0.49 | 2.30E+10 | 5.75E+09 | 103.20 ± 1.84 | 7.72E+04 |
| (80% | IM-300 μM | 18.40 | 6.13 | 7.64E+10 | 1.91E+10 | 111.40 ± 2.06 | 1.45E+05 |
| Amplitude) | IM-600 μM | 24.05 | 4.01 | 1.84E+10 | 4.60E+09 | 101.10 ± 2.27 | 7.87E+05 |
| Active loading | DH-300 μM | 9.98 | 3.33 | 8.38E+09 | 2.10E+09 | 128.50 ± 1.56 | 7.17E+05 |
| | DH-600 μM | 18.06 | 3.01 | 5.49E+09 | 1.37E+09 | 121.70 ± 0.82 | 1.98E+06 |
| | IM-300 μM | 26.01 | 8.67 | 6.68E+09 | 1.67E+09 | 121.30 ± 0.82 | 2.34E+06 |
| | IM-600 μM | 38.61 | 6.44 | 1.95E+09 | 4.88E+08 | 115.90 ± 6.38 | 1.19E+07 |
| Electroporation | DH-300 μM | 4.40 | 1.47 | 7.11E+10 | 1.85E+10 | 140.50 ± 0.43 | 3.73E+04 |
| | DH-600 μM | 4.47 | 0.74 | 3.70E+10 | 9.62E+09 | 134.80 ± 0.45 | 7.27E+04 |
| | DH-900 μM | 3.04 | 0.34 | 1.23E+10 | 3.20E+09 | 131.10 ± 0.76 | 1.49E+05 |
| | DH-3 μM | 4.01 | 0.13 | 2.82E+10 | 7.33E+09 | 135.70 ± 1.11 | 8.57E104 |
| | DH-6 μM | 2.39 | 0.04 | 5.28E+08 | 1.37E+08 | 100.30 ± 9.77 | 2.73E+06 |
| | DH-9 mM | 3.04 | 0.03 | 1.98E+08 | 5.15E+07 | 110.20 ± 11.52 | 9.25E+06 |
| | IM-300 μM | 22.88 | 7.63 | 3.61E+10 | 9.39E+09 | 131.60 ± 1.06 | 3.82E+05 |
| | IM-600 μM | 29.71 | 4.95 | 4.63E+10 | 1.20E+10 | 131.70 ± 0.91 | 3.86E+05 |
| | IM-900 μM | 12.66 | 1.41 | 7.56E+10 | 1.97E+10 | 136.60 ± 1.61 | 1.01E+05 |
| | IM-3 mM | 26.67 | 0.89 | 3.98E+10 | 1.03E+10 | 135.40 ± 0.52 | 4.04E+05 |
| | IM-6 mM | 246.34 | 4.11 | 6.38E+07 | 1.66E+07 | 155.60 ± 43.64 | 2.33E+09 |
| | IM-9 mM | 454.06 | 5.05 | 4.99E+07 | 1.30E+07 | 71.10 ± 51.76 | 5.48E+09 |

Drug encapsulation efficiency calculation:

This calculation quantifies how much drug is actually packaged with the EVs, compared to how much drug was input as starting material at the beginning of a drug packaging protocol.

(B/A)×100%

where,

### A=Starting Drug Concentration (μM)

B=Final drug concentration packaged with EVs after the entire packaging protocol and free drug clean-up (μM)
Drug molecules per EV:
This calculation quantifies the drug molecules packaged with each EV (average) at end of the packaging and clean-up process

(B*N)/(1000000000*X)

### Where,

X=Final EV Particle concentration (Particles/mL)
B=Final drug concentration packaged with EVs after the entire packaging protocol and free drug cleanup (μM)
N=6.022E+23 (Avogadro's number)

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-55. (canceled)

56. A composition comprising an isolated exosome(s) comprising a c-KIT inhibitor and a pharmaceutically acceptable excipient, wherein the c-kit inhibitor is sunitinib.

57. (canceled)

58. The composition of claim 56, wherein the composition comprises exosome(s) having a concentration of about 5 e+10 to 4e+11 exosome(s)/mL or about 83 pM to 0.66 nM.

59. (canceled)

60. The composition of claim 1, wherein less than about 5% of sunitinib in the composition is not associated with an exosome(s).

64. (canceled)
